(19) United States
(12) Patent Application Publication
(10) Pub. No.: US 2016/0242919 A1
(43) Pub. Date: Aug. 25, 2016

(54) APPARATUS AND METHOD FOR SCULPTING THE SURFACE OF A JOINT

(71) Applicant: Cayenne Medical, Inc., Scottsdale, AZ (US)

(72) Inventors: Gerard A. Engh, Remington, VA (US); Wesley D. Johnson, Eden Prairie, MN (US)

(21) Appl. No.: 15/146,582

(22) Filed: May 4, 2016

Related U.S. Application Data
(63) Continuation of application No. 11/458,942, filed on Jul. 20, 2006, now abandoned.
Application No. 11/458,942 is a continuation-in-part of application No. 11/186,485, filed on Jul. 20, 2005, now Pat. No. 7,758,652.
Application No. 11/186,485 is a continuation-in-part of application No. 10/159,147, filed on May 29, 2002, now Pat. No. 7,115,131.
Application No. 10/159,147 is a division of application No. 09/882,591, filed on Jun. 14, 2001, now Pat. No. 6,482,209.
(60) Provisional application No. 60/701,270, filed on Jul. 21, 2005.
(60) Provisional application No. 60/589,320, filed on Jul. 20, 2004.

(51) Int. Cl. A61F 2/38

(52) U.S. Cl. CPC A61F 2/3859; A61F 2/389; A61F 2002/3863; A61F 2002/30616

(57) ABSTRACT

The present invention provides a method and device for restoring individual patient joint kinematics using minimally invasive surgical procedures. The instrumentation of the invention sculpts the articular surface of a first bone that normally articulates in a predetermined manner with a second bone. The instrumentation includes a bone sculpting tool and a mount for attaching the tool to the second bone. The implant system is comprised of implants that provide intraoperative surgical options for articular constraint and facilitate proper alignment and orientation of the joint to restore kinematics as defined by the individual patient anatomy.

### CROSS REFERENCE TO RELATED APPLICATIONS

This application is a continuation of U.S. patent application Ser. No. 11/458,942, filed on Jul. 20, 2006; which claims the benefit of U.S. Provisional patent application Ser. No. 60/701,270, filed Jul. 21, 2005; and is a continuation-in-part of U.S. patent application Ser. No. 11/186,485, filed Jul. 20, 2005, now U.S. Pat. No. 7,758,652; which claims the benefit of U.S. provisional patent application Ser. No. 60/589,320, filed Jul. 20, 2004; and which is a continuation-in-part of U.S. patent application Ser. No. 10/159,147, filed May 29, 2002, now U.S. Pat. No. 7,115,131; which is a divisional of U.S. patent application Ser. No. 09/882,591, filed Jun. 14, 2001, now U.S. Pat. No. 6,482,209; the entireties of which are hereby incorporated by reference.

### BACKGROUND OF THE INVENTION

1. Field of the Invention

This invention relates to implants for use in minimally invasive total knee replacement surgery. More particularly, this invention relates to modular bearing surfaces and mobile-bearing and fixed-bearing modular components in arthroplasty of human joints.

2. Description of the Related Art

A joint, such as the ankle, knee, hip or shoulder, or a spinal motion segment generally consists of two or more relatively rigid bony structures that maintain a relationship with each other. Soft tissue structures spanning the bony structures hold the bony structures together and aid in defining the motion or kinematics of one bony structure to the other. In the knee, for example, the bony structures are the femur, tibia and patella. Soft tissue structures spanning the knee joint, or interposed, such as muscles, ligaments, tendons, menisci, and capsule, provide force, support and stability to facilitate motion or movement of the knee. Muscle and tendon structures spanning the knee joint, as in other joints of the body provide dynamics to move the joint in a controlled manner while stabilizing the joint to function in an orderly fashion. Dynamic stabilization is the result of primary muscle contraction to move the joint in a desired direction combined with antagonistic muscle contraction to direct resultant joint loads within favorable orientation limits relative to the bony structures of the joint. It is believed that proprioceptive feedback provides some of the control or balance between primary and antagonistic muscle contraction.

A smooth and resilient surface consisting of articular cartilage covers the bony structures of articular joints, whereas soft tissue structures, annulus and nucleus, provide motion between vertebral bodies. The articular surfaces of the bony structures work in concert with the soft tissue structures to form a mechanism that defines the envelop of motion between the structures. Within a typical envelop of motion, the bony structures move in a predetermined pattern with respect to one another, generally referred to as joint kinematics. When fully articulated, the motion defines a total envelop of motion between the bony structures. In the knee, the soft tissue structures spanning the joint in combination with articular geometry tend to stabilize the knee from excessive translation in the joint plane defined by the tibiofemoral joint. Such tibiofemoral stability enables the femur and tibia to slide and rotate on one another in an orderly or predetermined fashion. Similarly, the soft tissue structures of the joint capsule, the patellar ligament and the quadriceps tendon in combination with articular geometry tend to stabilize the patellofemoral joint from excessive mediolateral translation.

Current methods of preparing the intra-articular rigid elements of a joint to receive components as in joint replacement surgery involve an extensive surgical exposure. The surgical exposure, ligament release and sacrifice of the anterior cruciate ligament must be sufficient to permit the introduction of guides that are placed on, in, or attach to the joint, along with cutting blocks to guide the use of saws, burrs and other milling devices, and other instruments for cutting or removing cartilage and bone that subsequently is replaced with artificial surfaces. For knee joint replacement, the distal end of the femur may be sculpted to have flat anterior and posterior surfaces generally parallel to the length of the femur, a flat end surface generally normal to the anterior and posterior surfaces, and angled flat surfaces joining the above mentioned surfaces, all for the purpose of receiving a prosthetic device. In general these are referred to as the anterior, posterior, and distal and chamfer cuts, respectively.

In current total knee arthroplasty proper knee alignment is attained by preoperative planning and x-ray templating. Anterior-posterior (A/P) and lateral x-ray views are taken of the knee in full extension. The mechanical axis of the tibia and of the femur is marked on the A/P x-ray. The angle between these lines is the angle of varus/valgus deformity to be corrected. In the A/P view, the angle of the distal femoral resection is established relative to the femoral mechanical axis, hence the angle of the femoral implant is predetermined relative to the femur per the surgical technique for a given implant system. Similarly, the angle of the tibial resection is established relative to the tibial mechanical axis, hence the angle of the tibial implant is predetermined relative to the tibia per the surgical technique for a given implant system. The femoral resection guides are aligned on the femur to position the distal femoral resection relative to the femoral mechanical axis and the tibial resection guides are aligned on the tibia to position the proximal tibial resection relative to the tibial mechanical axis. If the cuts are made accurately, the femoral mechanical axis and the tibial mechanical axis will be properly aligned in the A/P view. For the patella, in general a planar resection is made at the articular margin; aligning the resection relative to the patella. This approach addresses knee alignment at full extension only. Knee alignment at 90.degree. of flexion is generally left to surgeon judgment and knee alignment throughout the range of motion has not been addressed in the past. In aligning the knee at 90.degree. the surgeon rotates the femoral component about the femoral mechanical axis to a position believed to provide proper tensioning of the ligaments spanning the knee.

Knee joint prosthesis of the type referred to above are well known, and are described, for example, in Caspari et. al., U.S. Pat. Nos. 5,171,244, 5,171,276 and 5,336,266, Brown, U.S. Pat. No. 4,892,547, Burstein et al., U.S. Pat. No. 4,298,992, and Insall et. al., U.S. Pat. No. 6,068,658.

Substantial effort has been made to provide appropriate degrees of curvature to the condyles in knee joint replacement. For example, the earlier mentioned U.S. Pat. Nos. 5,171,276, 4,298,992 and 6,068,658 show that the radius of curvature in the anterior-posterior direction of the condyle of a femoral prosthesis may be somewhat greater near the anterior portion of the condyle than near the posterior portion. Kester et al., U.S. Pat. No. 5,824,100 teaches that a portion of this curvature of the condyle may be formed about a constant radius having its origin along a line between the lateral and medial collateral ligament attachment points on the femur.

Historically, a variety of modular prosthetic joint implants have been developed. The following descriptions of modular implants relate specifically to the knee. Early designs for knee implants, called polycentric knee implants, were developed with separate components for the femoral and tibial surfaces of the medial and lateral tibiofemoral compartments. In this implant the patellofemoral compartment was not resurfaced. Orientating the separate components one to another, for example aligning the medial and lateral femoral components to one another, or the medial and lateral tibial components to one another, was not addressed in these designs and often left for the surgeon to make free hand resections resulting in a surgically challenging procedure. Designs emerged, such as the UCI and Gustilo knees in which the femoral condylar components were connected into an integral, unitary component as were the tibial components. The next advancement in total knee implant design was to include the patellofemoral joint by making an integral, unitary femoral component to resurface the medial and lateral femoral condyles and the femoral trochlea, commonly called the patellar groove. Implants to resurface the patella were developed in conjunction with the tri-compartmental femoral components. Additionally, modular fixed-bearing knee implants, generally referred to as semi-constrained, having a polyethylene insert that is held relatively rigidly in place have been developed. Translation and axial rotation between the tibia and femur that occurs naturally with knee motion is accommodated in these designs by non-conforming tibiofemoral contact for the medial and lateral condyles. Such designs tend to have higher contact pressure which may accelerate wear and degradation of the polyethylene bearing surface. Alternately, there are mobile bearing knee implants wherein the polyethylene bearing is structured to slide or move with minimal or no constraint on a tibial baseplate. These mobile bearing designs have high conformity between the polyethylene insert and femoral condyle and the polyethylene insert and tibial baseplate resulting in lower contact stresses and a more durable design. Furthermore, both meniscal bearing and fixed bearing knee implants have been developed including either separate polyethylene bearings in each of the medial and lateral tibiofemoral compartments, or a single polyethylene bearing spanning the medial and lateral tibiofemoral compartments that resides on a metallic tibial baseplate. While implant systems have been developed with fixed bearing elements or mobile bearing elements on the medial and lateral sides of the tibiofemoral joint, systems have not been developed having a combination of a fixed bearing on one side and a mobile bearing on the other side of the tibiofemoral joint.

Two primary difficulties exist with current joint replacement surgeries. These relate to the invasiveness of the procedure and achieving proper alignment and kinematics of the bony structures and the prostheses thereupon. Such difficulties are present in all total joint replacements, including but not limited to ankle, knee, hip, shoulder, wrist and finger. As well as in spinal disc replacement, nucleus replacement, facet joint replacement, or combinations thereof.

Alignment. A difficulty with implanting both modular and non-modular knee implants having either separate femoral and/or tibial components has been achieving a correct relationship between the components. Surgical instruments available to date have not provided trouble free use in implanting multi-part implants wherein the distal femur, proximal tibia and posterior patella are prepared for precise component-to-component orientation. While alignment guides aid in accurate orientation of opposing components relative to the axis of the long bones to achieve a restoration of a correct tibiofemoral varus/valgus alignment (usually 4-7 degrees valgus), they provide limited positioning or guidance relevant to correct subcomponent-to-subcomponent alignment in placing a plurality of components to form the articular surface of a femoral component or a tibial component. Such instrumentation references the bone on which it is placed and does not account for nor attempt to address ligament tension to restore soft tissue balance in a properly aligned total knee. Rather, such instrumentation relies on the surgeon to release ligaments and soft tissue structures to balance the knee and to accommodate positioning of the implants. For the patellofemoral joint, proper tibiofemoral alignment is required to re-establish proper tracking of the patella as created by the lateral pull of the quadriceps mechanism, the articular surface of the femoral patellar groove and maintaining the tibiofemoral joint line.

While surgical instruments available to date aid in accurate varus/valgus alignment, they provide limited positioning or guidance relevant to correct flexion/extension orientation of the femoral, posterior slope of tibial components, nor of external rotation of the femoral component. For optimum knee kinematics, femoral component flexion/extension and external rotation orientation, tibial component posterior slope and ligaments spanning the joint work in concert maintaining soft tissue balance throughout the knee's range of motion.

In a properly aligned knee, the mechanical axis of the leg (a straight line drawn from the center of the hip joint to the center of the ankle) passes slightly medial to the center of the knee. This alignment is generally called the gross alignment of the leg. The alignment of the implants impacts the gross alignment of the leg. If the implants are malaligned, the resulting mechanical axis may be shifted medially or laterally, resulting in an imbalance in the loads carried by the medial or lateral condyles. This imbalance, if severe, may lead to early failure of the arthroplasty.

In the case of a plurality of sub-components resurfacing the distal femur or proximal tibia the orientation of the sub-components to each other, for example the orientation of the medial femoral condylar sub-component to the femoral trochlear sub-component or to the lateral femoral condylar sub-component has largely not been addressed. Similarly for the tibial implant, orientation of the medial tibial sub-component to an independent lateral tibial sub-component has largely not been addressed. Moreover, orientation of the femoral component to the corresponding tibial component, whether with free standing uni-compartmental, bi-compartmental and/or tri-compartmental implants has largely not been addressed. This may account for the high failure rates in the surgical application of free standing compartmental replacements, used individually or in combination, and as well as for the higher failure rate of uni-compartmental implants relative to total knee implants as demonstrated in some clinical studies. When considering uni-compartmental designs the implant must be properly aligned and oriented with the ipsilateral condyle to maintain soft tissue structures spanning the knee in proper kinematic balance. Similarly, when considering bi-compartmental designs, alignment and orientation of each femoral sub-component to the other, or of each tibial sub-component to the other, is critical to maintain soft tissue structures spanning the knee in proper kinematic balance. In both case, as in the case of a tri-compartmental knee implant, proper sub-component to sub-component alignment and orientation is critical to avoid accelerated wear resulting from mal-articulation of the components.

Although various prosthetic devices have been successfully used with patients, the configuration and position of the articulating surfaces of the prosthesis, for example the condyles in a knee joint, are predetermined based upon the prosthesis that is selected. With a given knee implant system the implants are available in discrete sizes and the relationship, for example the ratio between medial-lateral width and anterior-posterior depth, vary between implant systems. While efforts are made to tailor the prosthesis to the needs of each patient by suitable prosthesis choice and size, this in fact is problematical inasmuch as the joint physiology of patients can vary substantially from one patient to another.

Invasiveness. In order to appropriately sculpt the articulating surface of a bone, it is often necessary to surgically expose the joint. In the case of the femur in traditional knee joint replacement, the patellar tendon of the knee joint is surgically exposed and is moved to one side of the joint and the patella everted to enable a substantially full anterior access to the joint. In general, the anterior cruciate ligament is excised to increase access to the joint space. Surgical exposure is necessary to accommodate the bulk and geometry of the components as well as the instruments for bone preparation. Such surgical exposure and ligament release or excision increases bleeding, pain, muscle inhibition and adverse kinematics; all of which contribute to a longer hospitalization before the patient can be safely discharged to home or an intermediate care facility. Altered kinematics can reduce a patient's confidence in the knee's ability to perform demanding tasks, and at times tasks of daily living, to the point of significantly limiting lifestyle and activity level.

Desirably, in the case of knee replacement surgery, neither the collateral ligaments nor the cruciate ligaments are disturbed, although it is often necessary to remove or release cruciate ligaments in the event a substantial joint replacement is to be performed. Collateral ligaments can be partially taken down or released to provide appropriate tension adjustment to the patient's knee in concert with joint replacement surgery. In most instances, such releases can be accomplished through smaller incisions than the standard midline or medial parapatellar incisions historically used for knee arthroplasty.

For patients who require articular surface replacement, including patients whose joints are not so damaged or diseased as to require whole joint replacement, the implant systems available for the knee have unitary tri-compartmental femoral components, unitary tibial components, unitary patellar components and instrumentation that require extensive surgical exposure to perform the procedure. It would be desirable to provide surgical methods and apparatuses that may be employed to gain surgical access to articulating joint surfaces, to appropriately prepare the bony structures, to provide artificial, e.g., metal, plastic, ceramic, or other suitable material for an implant or articular bearing surface, and to close the surgical site, all without substantial damage or trauma to associated muscles, ligaments or tendons, without extensive distraction of the joint, and without disruption of the patient's normal kinematics. To attain this goal, implants and instruments are required to provide a system and method to enable articulating surfaces of the joints to be appropriately sculpted using minimally invasive apparatuses and procedures and to replace the articular surfaces with implants suitable for insertion through small incisions, assembly within the confines of the joint cavity and conforming to prepared bone support surfaces.

### SUMMARY OF THE INVENTION

The present invention provides a system and method for total joint replacement that is to resurface each bony surface of the joint or motion segment that involves minimally invasive surgical procedures including an implant system that restores individual patient's joint kinematics. A feature of the invention is engaging or joining the plurality of sub-components comprised in an implant system, for example a knee implant system. Another feature of the invention is instrumentation to simplify accurate and repeatable placement of the plurality of sub-components comprised in an implant system. As used herein, the following terms have the following definitions.

Minimally invasive or less invasive—For the purposes of the current invention as applied to knee arthroplasty an incision for conventional total knee arthroplasty is defined as being generally greater than 6 inches in length. An incision for minimally and less invasive knee arthroplasty is defined as being generally less than 6 inches in length.

Engage—For the purposes of the current invention engage pertains to 1) engagement of sub-components of an implant to form the implant, and 2) engagement of implant components of a joint arthroplasty. In both cases engage means to cause mechanical parts (i.e. sub-components of a femoral component for example, or a set of components to include femoral, tibial and patellar components for example) to come together, to mesh to one another, or to come into working contact with one another. Such contact between adjoining parts limiting at least one degree of freedom between the parts.

Joining—For the purposes of the current invention joining pertains to joining of sub-components of an implant to form the implant and means to cause mechanical parts (i.e. sub-components of a femoral component for example) to be interlocked together and constrained in one or more degrees of freedom so as to form a unit.

Orienting—For the purposes of the current invention orientating pertains to 1) orientating sub-components of an implant to one another, and 2) orientating implant components of a joint arthroplasty to one another. In both cases orientating means to bring the parts into working relationship to one another so that the assembly of parts functions as intended.

Aligning—For the purposes of the current invention aligning pertains to 1) alignment of sub-components of an implant to supporting bone, and 2) alignment of implant components of a joint arthroplasty to supporting bone. In both cases aligning means to bring the parts into correct relative position with respect to the supporting bone so that the arthroplasty functions as intended.

Implant component and sub-component—For the purposes of the current invention an implant component refers to the parts that make up the arthroplasty, for example femoral, tibial and patellar components make up a total knee arthroplasty. Sub-component refers to the parts that make up the implant component. Each component may be unitary in construction, or may include a plurality of sub-components.

For the purposes of describing the invention, arthroplasty includes total and partial joint replacement (i.e. hip, knee, shoulder, ankle, finger joints, etc.) and total and partial spinal disc and facet replacement. Such arthroplasty systems include components such as femoral, tibial and bearing insert(s) components for a knee arthroplasty; stem, head, bearing insert and shell components for a hip arthroplasty; and vertebral endplates and bearing insert(s) and facet joint replacements for spinal arthroplasty.

Sequence of assembly of sub-components and placement onto supporting bone—For the purposes of the current invention the sequence of assembly of sub-components and placement onto supporting bone may be varied. That is to say, the sub-components may be a) partially assembled outside the joint cavity, passed into the joint cavity, assembled and placed onto the supporting bone; b) individually passed into the joint cavity, assembled and placed onto the supporting; c) individually passed into the joint cavity, placed onto the supporting bone and assembled thereon; d) individually passed into the joint cavity, one or more sub-components attached to supporting bone, then one or more of the remaining sub-components assembled to those previously attached to bone; or e) any combination thereof.

The instruments and implants disclosed accomplish accurate bone and soft tissue preparation, restoration of anatomical alignment, soft tissue balance, kinematics, component to component orientation and alignment, sub-component to sub-component orientation and alignment, and implant fixation to supporting bone through limited surgical exposure. For knee joint replacement, the implant system is comprised of implants that provide intraoperative surgical options for articular constraint and facilitate proper alignment and orientation of the knee to restore anatomical alignment, soft tissue balance and kinematics as defined by individual patient anatomy. To do so, the implants provide the surgeon intraoperative options to reconstruct various degrees of joint stability via selection of fixed or mobile bearing components for each compartment of the knee (medial tibiofemoral, lateral tibiofemoral and patellofemoral compartments). The range of implants may be applied to one, two or three of the knee joint compartments in a given procedure and may include combinations of fixed and mobile bearing configurations.

In conventional total knee replacements, the femoral component is typically a unitary piece and the tibial baseplate component is a unitary piece. A bearing is placed between the femoral and tibial baseplate components which is generally a unitary piece that may be fastened to the tibial component or sliding on the tibial baseplate component. In the present invention, the femoral side may be resurfaced by two, three or more individual sub-components and the tibial side may be resurfaced by two or more tibial baseplate sub-components or a unitary baseplate. Alternatively, the femoral side may be resurfaced with a component of unitary structure and the tibial side may be resurfaced by two or more tibial baseplate sub-components. The modular femoral component comprised of two or more sub-components is sized to be placed through a minimally invasive incision into the joint space one piece at a time and assembled therein during the surgical procedure. Likewise, the modular tibial component comprised of one or two polyethylene bearings and a baseplate component comprised of two or more individual sub-components each of which is sized to be placed through a minimally invasive incision into the joint space one piece at a time and assembled therein during the surgical procedure.

Alternatively, the multi-piece tibial component may have a stem that can be placed individually into the joint space and structured to pass down the tibial medullary canal and assemble to the baseplate or baseplate sub-components within the confines of the joint space. Likewise, the modular femoral component may have a stem that can be placed individually into the joint space and structured to pass down the femoral medullary canal and assemble to the femoral sub-components.

The femoral sub-components are accurately aligned to supporting bone and orientated to one another with or without interconnecting the individual sub-components after placement in the joint cavity. Likewise, the tibial sub-components are accurately aligned to supporting bone and orientated to one another with or without interconnecting the individual sub-components once placed in the same manner. In both cases, the size of each component or sub-component passed into the joint is significantly reduced compared to conventional components enabling completion of the procedure through a smaller and less traumatic exposure.

In the case of interconnected sub-components, comprising the femoral component, the tibial component, or both, such interconnection may be structured as an engaging mechanism between adjacent sub-components, or as a joining mechanism between adjacent sub-components. In the case of three of more sub-components, a combination of engaging or joining mechanisms may be used between various adjacent sub-components. Optionally, such engaging or joining between adjacent sub-components may be temporary during the surgical procedure to aid in orienting the sub-components while securing them to supporting bone. The patellar component is generally of a size that can be placed through minimally invasive incisions as a unitary bearing, fixed bearing or mobile bearing component. In one aspect of the present invention, the articular surface of the patellar component may comprise independent, individual sub-components for the lateral facet and medial facets which are properly orientated, but not joined within the joint cavity. In yet another aspect of the present invention, the independent patellar sub-components may be properly orientated and joined within the joint cavity. In still another aspect of the present invention, the femoral component may be flexible or include flexible sub-components.

The femoral, tibial and patellar components of the present invention as used in partial or total knee arthroplasty are structured to have one surface for bony attachments. Such attachment provided by a porous or roughened surface into which, or onto which, the supporting bone can grow. Alternatively, such attachment is provided by a porous or roughened surface into which, or onto which, bone cement can attach. In yet another embodiment the surface of the sub-component in contact with supporting bone is coated with a biological adhesive or bone growth factor to provide initial stability and to promote rapid bony integration.

Proper alignment and orientation of the implant components and sub-components may be enabled by instruments guided by the soft tissue structures of the knee to guide bone resections for patient-specific anatomical knee alignment and component and sub-component orientation. The medial and lateral tibial articular surfaces and the patellar articular surface are generally prepared with planar resections. The medial and lateral femoral condyles and trochlea may be kinematically prepared. Such instrumentation is referred to as Tissue Guided Surgery (TGS) and is described in U.S. Pat. No. 6,723,102 and is incorporated by reference in its entirety. Alternatively, the medial and lateral femoral condyles and trochlea may be prepared with planar resections and chamber resection as is typical in conventional total knee arthroplasty. Such preparation is possible with conventional total knee instrumentation as is commonly known by those skilled in the art. Alternatively, such preparation is possible with Tissue Guided Surgery by positioning the tibia with a bone sculpting tool at appropriate knee flexion angles to sculpt planar resections for the posterior, posterior chamfer and distal femoral resections, and by positioning the patella at appropriate knee flexion angles to sculpt planar resections for the anterior chamfer and trochlear resections. Hence, the present invention for joining or engaging the plurality of sub-components comprised in a knee implant system and instrumentation to simplify accurate and repeatable placement of the plurality of sub-components comprised in a knee implant system is applicable to conventional knee implants.

Femoral, tibial and patellar bone resections attained with TGS instrumentation are properly positioned and orientated for anatomic knee alignment, soft tissue balance and kinematic function throughout knee range of motion. Using these bone support surfaces to position and orientate the femoral, tibial and patellar components, respectively, will maintain anatomic knee alignment, soft tissue balance and kinematic function. In general, the tibial and patellar resections are planar making placement of the corresponding implant components, which have planar support surfaces, straight forward. The femoral resection may not be planar if the supporting bone is prepared via TGS and the relative position of the lateral condyle, the medial condyle and the trochlear resections to one another is a function the kinematics of a given patient. Therefore, the femoral implant must accommodate this variability, as described herein.

Given that the soft tissue structures spanning the knee are used to guide the TGS instrumentation it is beneficial for such tissues to be minimally disrupted by the surgical technique and to avoid dislocation or eversion of the patella. The minimally invasive surgical incision or incisions used to access the knee joint must be of a size and orientation relative to the soft tissue structures that minimizes alteration of knee kinematics. The femoral, tibial and patellar implants must be structured to pass through such minimally invasive incisions. Conventional femoral and tibial implants for total knee arthroplasty are sized so large that insertion through a minimally invasive incision is not feasible. In addition, the shape of conventional femoral components does not permit placement of the component over the resected distal femur with the majority of soft tissues intact or without dislocation or eversion of the patella. Further, the confines of the joint cavity do not provide sufficient space to align conventional femoral components distal to the anterior and posterior femoral resections and then slide the component over those resections. Therefore, the femoral, tibial and patellar components must be sized to be passed through a small incision and to be placed onto or over the respective bone support surfaces. For the femoral component, one embodiment is a component made up of a plurality of sub-components to resurface the medial condyle, the lateral condyle and the trochlea of distal femur. Such sub-components are of a size that can be passed through a small incision and be assembled, that is to be joined or engaged, within the confines of the joint cavity. Optionally, such joining or engaging between adjacent sub-components may be temporary during the surgical procedure to aid in orienting the sub-components while securing them to supporting bone.

Femoral sub-components conform to the shape of the kinematically prepare condyles and trochlea. The interfaces between femoral sub-components are partially constrained. These interfaces are unconstrained in angulation generally in a sagittal plane to allow the sub-components to conform to the trochlear and condylar resections. These interfaces are constrained in angulation generally in a transverse plane, in orthogonal and axial translation and in axial rotation to provide a smooth transition from one sub-component to an adjacent sub-component. A smooth transition provides uniform support for the mating tibial or patellar component. Alternatively, the interfaces between the femoral sub-components are unconstrained in angulation and constrained in other degrees of freedom to allow the femoral component to conform to the resected femoral condyles and to vary the anteroposterior divergence of the condylar sub-components with a similar divergence in tibial sub-components. Alternatively, the interfaces between the femoral sub-components are fully constrained when fully assembled. Likewise, tibial sub-components are properly aligned one to the other to ensure proper tracking of the femoral, tibial and patellar components. The tibial sub-components may be constrained or unconstrained one to the other in similar fashion as that described above for femoral sub-components.

In addition to preparing the bone for patient-specific alignment and orientation of the implant components, the present invention provides further component orientation by joining or engaging the femoral sub-components together and joining or engaging the tibial sub-components together. The femoral sub-components may be temporarily or permanently joined after being placed into the joint space. Likewise, the tibial sub-components may be temporarily or permanently joined after being placed into the joint space. When the sub-components are to be temporarily joined within the joint space one or more brackets are interposed between the sub-components and temporarily secured or assembled to each sub-component. The brackets hold the sub-components in proper alignment and orientation to each other while the component is secured to bone by mechanical means such as bone screws, spikes, hooks, etc., or bone cement, or other bonding material or process. The bracket or brackets may be of rigid construction being made from metals, such as stainless steels, cobalt chromium alloys, titanium or titanium alloys, ceramics or other suitable materials; or rigid plastics such as PEEK or other suitable plastics. Alternatively, the bracket or brackets are of flexible construction being made of metals such as Nitinol, NP35N, or other suitable materials; flexible plastics such as UHMW Polyethylene or urethane; or woven materials such as Gore-Tex or other suitable materials. At each juncture between sub-components the bracket is structured to maintain a smooth transition of the articular surface between adjacent sub-components while enabling each sub-component to conform to the bony support surface. The bracket or brackets are removed after the components are secured to the supporting bone. Removal of the temporary brackets may be at the time of surgery, or at some later date. Alternatively, the brackets may be structured as an implant sub-component to remain implanted.

In the case of knee replacement surgery, the implants include a second bone baseplate, a bearing insert and a first bone implant. The second bone baseplate may be either one piece to generally cover the prepared surface of the second bone as relates to the joint, or separate baseplates as have been used with mobile or fixed bearing prosthetic components. Optionally, the one piece baseplate or the plurality of baseplate sub-components may be structured for assembly to a stem sub-component within the confines of the joint cavity. For either the one piece baseplate or the plurality of baseplate sub-components the bearing insert may be of unitary structure. Alternately, the bearing insert may be separate inserts. In addition, the second bone baseplate component may accommodate separate fixed and mobile bearing inserts used in medial and lateral combinations of fixed-fixed, mobile-fixed, fixed-mobile and mobile-mobile bearing inserts, respectively.

When assembling a plurality of sub-components, either to form a femoral component or a tibial component, within the confines of the joint cavity it is beneficial to structure the engaging or joining mechanisms to allow angulation and translation between the sub-components during assembly, then when fully assembled structure the engaging or joining mechanisms according to the constraints required for the femoral or tibial component. Such angulation and translation between adjacent sub-components during assembly being unconstrained or partially constrained as appropriate to make assembly of the sub-components as easy as possible for the surgeon. Such constraints for the fully assembled component to include unconstrained, partially constrained and fully constrained engaging or joining mechanisms between two or more sub-components, and combinations of unconstrained, partially constrained or fully constrained engaging or joining mechanisms connecting the plurality of sub-components that form an implant component.

The current invention is structured to allow variation in the procedure for implanting a plurality of sub-components forming either a femoral or tibial component. In general, the femoral component is implanted before the tibial component because the space within the joint cavity is more limited after placement of one of the components. The general shape of the femoral sub-components is bulkier than that of the tibial baseplate sub-components, hence the benefit of implanting them first. Alternatively, the tibial sub-components may be implanted first. In an alternative embodiment of the present invention that includes a tibial stem sub-component or a femoral stem sub-component, or both, it may be beneficial to place the stem sub-component(s) first, either into the femoral canal, into the tibial canal or into both. Followed by placement of the femoral condylar sub-components and trochlear sub-component, and then placement of the tibial baseplate sub-components and bearing insert(s). Alternatively, the femoral condylar sub-components and trochlear sub-component may be implanted first, followed by placement of the tibial baseplate sub-components, and then placement of a femoral stem sub-component or a tibial stem sub-component or both followed by placement of the bearing insert(s). In general, the patellar component is implanted last. Alternatively, one or more of the femoral sub-components or the tibial sub-components may be secured to supporting bone before assembly to respective adjacent sub-components. It may also be advantageous to partially assemble femoral sub-components or tibial sub-components outside of the joint cavity, for example passing the femoral medial condylar sub-component into the joint cavity then assembling the lateral condylar sub-component to the trochlear sub-component and passing the assembly into the joint cavity for assembly to the medial condylar sub-component.

In the case of tri-compartmental knee arthroplasty the articular surfaces of the tibia and patella are generally removed with planar resections which in general have minimal regional variations in the contour of the planar resections; however in preserving the anterior cruciate ligament it may be advantageous to resect the medial and lateral tibial articular surfaces independently which may result in variations between the planar resection of the medial tibial articular surface and that of the lateral tibial articular surface. The articular surfaces of the distal femur, those being the medial and lateral condyles and the trochlea, may be independently sculpted. The regional contour of the supporting bone, that is the contour of the resected bone within each compartment, that is the medial tibiofemoral compartment, the lateral tibiofemoral compartment and the patellofemoral compartment, closely matches that of the respective sub-component; however due to the independent sculpting of the femur within each compartment there may be variations between the prepared bone surfaces in each compartment. Additionally, partial constraint of the assembled interface between sub-components promotes load sharing across all resected surfaces of the supporting bone.

Means for joining partially constrained interfaces between sub-components includes, but is not limited to, spherical, meshed, cylindrical, planar, linear and point contact interfaces; “T” slots; dovetail locks; cylindrical interlocks; button interlocks; spherical interlocks; or a combination of these, or other connecting means used to connect two or more parts. Means for joining fully constrained interfaces between sub-components includes, but is not limited to, threaded fasteners, cylindrical pins, conical taper locks, square or rectangular taper locks, tether cable or wire locks, or a combination of these, or other fastening means used to connect two or more parts.

In the case of independent baseplate sub-components that are not joined together, it is beneficial to have a bracket that attaches to individual sub-components to hold them properly orientated one to another while they are secured to the supporting bone. Means to attach the bracket to the baseplate sub-components includes threaded fasteners, clamping devices, dovetails, trinkle locks, tether cable or wire attachments, or a combination of these, or other fastening means used to connect two or more parts. Optionally, a handle may be structured to attach to the bracket to simplify placement of the sub-components into the joint cavity.

The first bone implant is comprised of a plurality of sub-components to replace the bearing surface of the first bone. In the case where the sub-components the femoral component are properly orientated and joined within the joint, fastening means used to join the individual sub-components together include threaded fasteners, cylindrical pins, conical taper locks, square or rectangular taper locks, tether cable or wire locks, a combination of the foregoing, or any such other fastening means that can be used to connect two or more parts. In the case where the sub-components are not joined together, it is beneficial to have a bracket that attaches to the sub-components to hold them properly orientated one to another while they are secured to the supporting bone. Means to attach a bracket to the sub-components includes threaded fasteners, clamping devices, dovetails, trinkle locks, tether cable or wire attachments, or a combination of these, or other fastening means used to connect two or more parts.

Specifically, for example in knee joint replacement, the invention may be used for replacing the surfaces of a femur, a tibia, a patella, or a combination of these. Thus, a femoral implant having a plurality of sub-components, a tibial baseplate having a plurality of sub-components and a patellar component having a plurality of sub-components are provided. The tibial baseplate components and the patellar components may have fixed bearing attachments as well as mobile bearing attachments. Optionally, each component of the tibial baseplate or patellar may have a fixed bearing attachment as well as a mobile bearing attachment. Alternatively, the tibial component and the bearing attachment may be of unitary construction and the patellar component and bearing attachment may be of unitary construction. Optionally, the femoral and tibial components of the invention may be used with modular femoral and tibial stems, respectively.

The present invention for joining or engaging a plurality of sub-components comprised in a knee implant system and the instrumentation to simplify accurate and repeatable placement of the plurality of sub-components comprised in a knee implant system are applicable to the femoral, tibial, patellar and bearing insert components of a knee implant. In addition, this embodiment of the present invention is applicable to other joint implants, including but not limited to hip, shoulder, fingers and ankle; to spinal implants including but not limited to spinal disc replacement, facet replacement and spinal fusion; and to orthopaedic trauma products to include but not limited to fracture fixation systems.

### BRIEF DESCRIPTION OF THE DRAWINGS

FIG. 1 is a plane view of a knee joint.

FIG. 2 illustrates a traditional midline incision for accessing the knee joint during total knee replacement surgery.

FIG. 3 depicts an incision for accessing the knee joint during total knee replacement surgery that may be used with the method and apparatus of the present invention.

FIG. 4 illustrates alternate incisions for accessing the knee joint during total knee replacement surgery that may be used with the method and apparatus of the present invention.

FIG. 5 is a plane view of femoral resections made in accordance with an embodiment of the present invention.

FIG. 6 is a plane view of femoral resections made in accordance with an alternate embodiment of the present invention containing femoral implants.

FIG. 7 is a plane view of femoral resections made in accordance with a yet another embodiment of the present invention containing femoral implants.

FIG. 8 are plane views of alternate embodiments of tibial baseplates in accordance with an embodiment of the present invention.

FIG. 9 is a plane view of femoral implants for resurfacing the femoral resections of FIG. 6 according to an embodiment of the present invention.

FIG. 10 is a plane view of femoral implants for resurfacing the femoral resections of FIG. 7 according to an embodiment of the present invention.

FIG. 11 is a plane view of a femoral implant in accordance with an embodiment of the present invention.

FIG. 12 is an end view of femoral implants for the medial and lateral condylar sub-components and the trochlear sub-component according to embodiment of the present invention.

FIG. 13 is an end view of femoral implants for the medial condylar sub-component and the unitary lateral condylar and trochlear sub-component according to embodiment of the present invention.

FIG. 14 is an end view of femoral implants for the medial and lateral condylar sub-components and the trochlear sub-component according to embodiment of the present invention.

FIG. 15 is an end view of femoral implants for the medial condylar sub-component and the unitary lateral condylar and trochlear sub-component according to embodiment of the present invention.

FIG. 16 is an end view of femoral implants for the medial and lateral condylar sub-components and the trochlear sub-component according to embodiment of the present invention.

FIG. 17 illustrates femoral, tibial and patellar implants according to an embodiment of the present invention.

FIG. 18 illustrates femoral, tibial and patellar implants according to another embodiment of the present invention.

FIGS. 19 A & B are orthogonal views, one exploded and one assembled respectively, of the tibial inserter instrument according to an embodiment of the present invention.

FIGS. 20A & B are orthogonal views, one exploded and one assembled respectively, of the femoral inserter instrument according to an embodiment of the present invention.

FIG. 21 is a side view of a femoral component on a prepared femur according to an embodiment of the present invention.

FIG. 22 is an orthogonal view of a femoral component with a condylar sub-component according to an embodiment of the present invention.

FIG. 23 is a plane view of FIG. 22 according to an embodiment of the present invention.

FIG. 24 is an orthogonal view of a femoral component with two condylar sub-components according to an embodiment of the present invention.

FIG. 25 is a plane view of FIG. 24 according to an embodiment of the present invention.

FIG. 26 is an orthogonal view of a femoral component with a condylar sub-component comprised of the medial and lateral femoral condyles according to an embodiment of the present invention.

FIG. 27 is a plane view of FIG. 26 according to an embodiment of the present invention.

FIG. 28 is an orthogonal close-up view of an interface between femoral sub-components according to an embodiment of the present invention.

FIG. 29 is a plane view of FIG. 28 according to an embodiment of the present invention.

FIG. 30 is an orthogonal close-up view of another interface between femoral sub-components according to an embodiment of the present invention.

FIG. 31 is a plane view of FIG. 30 according to an embodiment of the present invention.

FIGS. 32 A & B are orthogonal views of yet another interface between femoral sub-components according to an embodiment of the present invention.

FIG. 33 is a cross sectional view of FIG. 32 according to an embodiment of the present invention.

FIGS. 34 A & B are orthogonal views of an interface between femoral sub-components according to an embodiment of the present invention.

FIG. 35 is a cross sectional view of FIG. 34 according to an embodiment of the present invention.

FIGS. 36 A & B are orthogonal views of another interface between femoral sub-components according to an embodiment of the present invention.

FIG. 37 is a cross sectional view of FIG. 36 according to an embodiment of the present invention.

FIGS. 38 A & B are orthogonal views of yet another interface between femoral sub-components according to an embodiment of the present invention.

FIG. 39 is a schematic of an interface bracket to hold implant sub-components together according to an embodiment of the present invention.

FIG. 40 is a cross sectional view of FIG. 38 according to an embodiment of the present invention.

FIG. 41 is a cross sectional view of a constrained interface between tibial sub-components according to an embodiment of the present invention.

FIG. 42 is a cross sectional view of another constrained interface between tibial sub-components according to an embodiment of the present invention.

FIG. 43 is a plane view of a tibial implant with unitary baseplate according to an embodiment of the present invention.

FIG. 44 is an orthogonal view of a tibial implant with a two piece joined baseplate according to an embodiment of the present invention.

FIG. 45 is an orthogonal view of a tibial implant with a unitary baseplate joined to a stem according to an embodiment of the present invention.

FIG. 46 is an exploded view of FIG. 45 according to an embodiment of the present invention.

FIG. 47 is an orthogonal view of another tibial implant with a unitary baseplate joined to a stem according to an embodiment of the present invention.

FIG. 48 is an orthogonal view of a femoral implant with trochlear, medial condylar and lateral condylar sub-components according to an embodiment of the present invention.

FIGS. 49 A and B are orthogonal views of a femoral component according to an embodiment of the present invention.

FIG. 50 is an orthogonal view of a tibial implant with unitary stem and baseplate covering one compartment of the tibial plateau, and a baseplate sub-component to cover the ipsilateral compartment of the tibial plateau according to an embodiment of the present invention.

FIGS. 51 A & B are orthogonal views of a tibial implant according to an embodiment of the present invention.

FIG. 52 is a side view of a femoral component on a prepared femur according to an embodiment of the present invention.

FIG. 53 is a cross sectional view of FIGS. 49 A and B of a femoral component according to an embodiment of the present invention.

FIG. 54 is a cross sectional view of a femoral component according to an embodiment of the present invention.

FIG. 55 is an exploded view of the tibial inserter instrument with an alignment guide according to an embodiment of the present invention.

FIG. 56 is an exploded view of the femoral inserter instrument with an alignment guide according to an embodiment of the present invention.

FIG. 57 is an exploded view of the tibial inserter instrument with a surgical navigation tracker according to an embodiment of the present invention.

FIG. 58 is an exploded view of the femoral inserter instrument with a surgical navigation tracker according to an embodiment of the present invention.

### DETAILED DESCRIPTION OF THE INVENTION

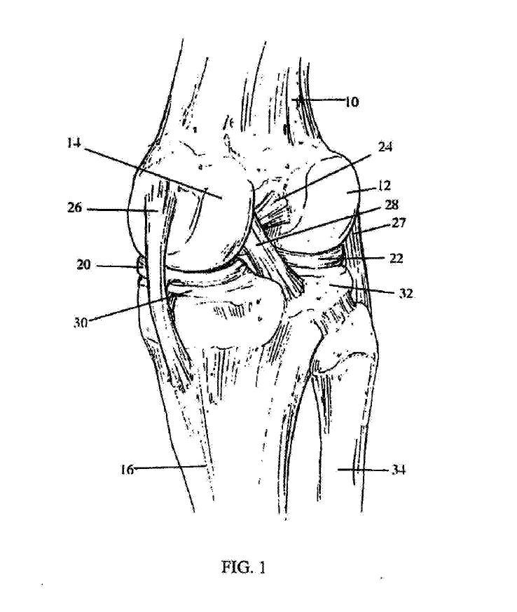

Knee Joint Anatomy and Surgical Approaches. FIG. 1 illustrates the general anatomy of the knee joint. The femur 10 has the lateral femoral condyle 12 and the medial femoral condyle 14 on its knee-joint articulating surface. The tibia 16 has the lateral meniscus 22 (generally opposite the lateral femoral condyle 12) and the medial meniscus 20 (generally opposite the medial femoral condyle 14) on its knee-joint articulating surface. The ligaments include the anterior cruciate ligament 24, the posterior cruciate ligament 28, the medial collateral ligament 26 and the lateral collateral ligament 27. The medial tibial condyle 30 and the lateral tibial condyle 32 support the menisci 20 and 22, which in turn support the femur 10. Additionally, the fibula 34 engages the tibia 16.

Typically, a total knee joint replacement involves replacing the articular surfaces of the lateral femoral condyle 12, the medial femoral condyle 14, the medial tibial condyle 30 and the lateral tibial condyle 32. The lateral meniscus 22 and the medial meniscus 20 are removed. Desirably, neither the collateral ligaments 26 and 27 nor the cruciate ligaments 24 and 28 are disturbed. However, the collateral ligaments 26 and 27 may be partially taken down to provide appropriate tension adjustments to the patient's knee after joint replacement has been completed. Such structures are contained within the intact knee joint cavity which is formed by the knee synovial bursa (not shown).

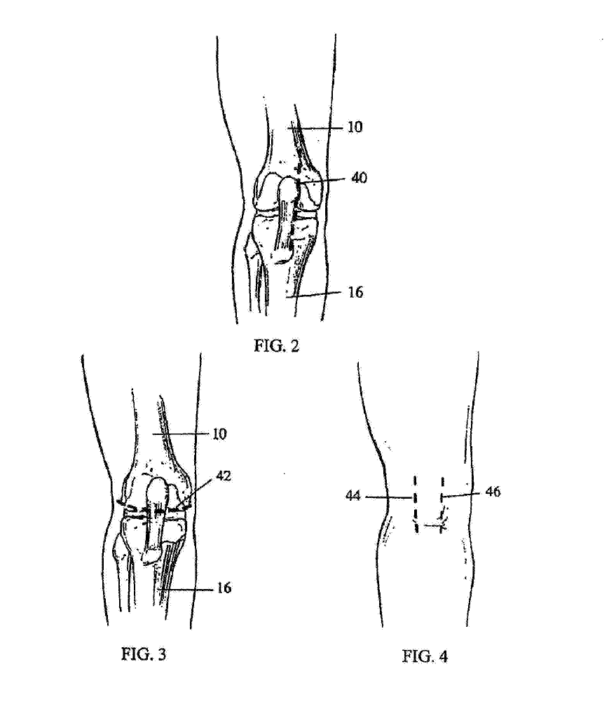

Referring to FIG. 2, the conventional midline incision 40 for a total knee replacement surgery is shown. The incision 40 extends vertically substantially above and below the articulating surface between the femur and the tibia. Typically, the incision is roughly 8 to 15 centimeters in length. The incision 40 must be large enough to expose the entire knee joint articular surfaces with the patella subluxed or dislocated. Additionally, the incision must accommodate insertion of components that fully cover the end of the femur, the top of the tibia and the undersurface of the patella. The maximum number of components implanted would include femoral and tibial components for the lateral tibiofemoral compartment, femoral and tibial components for the medial tibiofemoral compartment and femoral and patellar components for the patellofemoral joint. Alternatively, the lateral femoral condyle and the patellar groove may be covered by a common implant. The knee joint cavity is substantially opened by the incision 40 and the exposed articular surfaces of the knee protrude out of the joint cavity to accommodate current bone resection instruments and insertion of components that fully cover the end of the femur, the top of the tibia and the undersurface of the patella.

As best seen in FIG. 3, a transverse incision 42 extending horizontally along the knee joint is one option for the procedure of the present invention. The incision 42 may be vertically opened to expose the joint surfaces of the medial tibiofemoral compartment and the lateral tibiofemoral compartment without dislocating the patella. This maintains the patella in contact with the femur during the procedure. The components of the instrumentation as well as the implant are sized for minimal invasiveness and, therefore, may be accommodated by the small incision. The reduced trauma resulting from a smaller incision generally results in faster and better rehabilitation, which in turn generally increases the efficacy of the knee implant.

Referring to FIG. 4, an alternate incision format for use with the present invention is shown. Two parallel vertically extending incisions 44 and 46 may be formed on either side of the patella. These incisions 44 and 46 are relatively short and the invasiveness is similar to that of the horizontal incision in FIG. 3. Each incision 44 and 46 is separately extended through the joint capsule to expose the medial and lateral tibiofemoral compartments without dislocating the patella. In a one embodiment of the present invention the procedure is carried out through one small incision 46 medial to the patella.

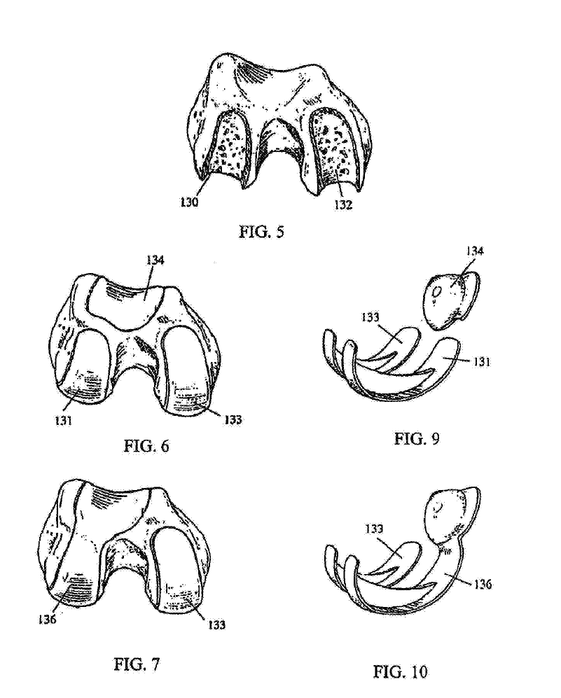

The femoral condyles may be prepared independent of the femoral trochlea as shown in FIG. 5. The lateral condylar resection 130 and the medial condylar resection 132 extend throughout the range of tibiofemoral contact resulting from flexing and extending the knee with a sculpting tool placed on the femur. Once prepared, the condylar resections receive a lateral condylar sub-component 131 and a medial condylar sub-component, respectively, and a femoral trochlear sub-component 134, each of which is shown as unconstrained relative to the adjacent sub-component, as shown in FIG. 6. In an alternate embodiment of the present invention the lateral condylar and the femoral trochlear resurfacing implants are constructed in a unitary sub-component 136 that resurfaces the lateral condyle and trochlea as shown in FIG. 7. The medial condylar sub-component 133 is independent and unconstrained relative to the lateral condylar-trochlear sub-component. Optionally, the lateral condylar-trochlear sub-component 136 may be implanted with an intact medial condyle, forgoing preparation and resurfacing of the medial condyle. Alternatively, the medial condylar and femoral trochlear resurfacing implants may be constructed in a unitary sub-component that resurfaces the medial condyle and the femoral trochlea. In which case the lateral condylar sub-component is independent of the medial condylar-trochlear sub-component. Optionally, the medial condylar-trochlear sub-component may be implanted with an intact lateral condyle, forgoing preparation and resurfacing of the lateral condyle.

The surgical procedure may be performed through one or more minimally invasive incisions that do not necessitate subluxation or dislocation of the patella. Therefore, implants such as the femoral, tibial or patellar implants are structured to fit through minimally invasive incisions, conformed to the kinematically prepared bone support surfaces, and aligned and oriented, and engaged or joined within the knee joint. The femoral and tibial implants may be attached to bone with conventional bonding methods such as, but not limited to, polymethylmethacrylate, or by direct attachment to bone as with, but not limited to, a porous ingrowth surface.

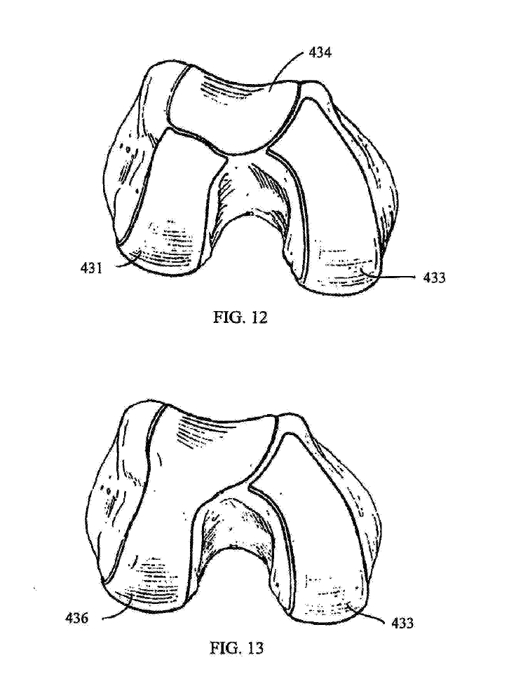

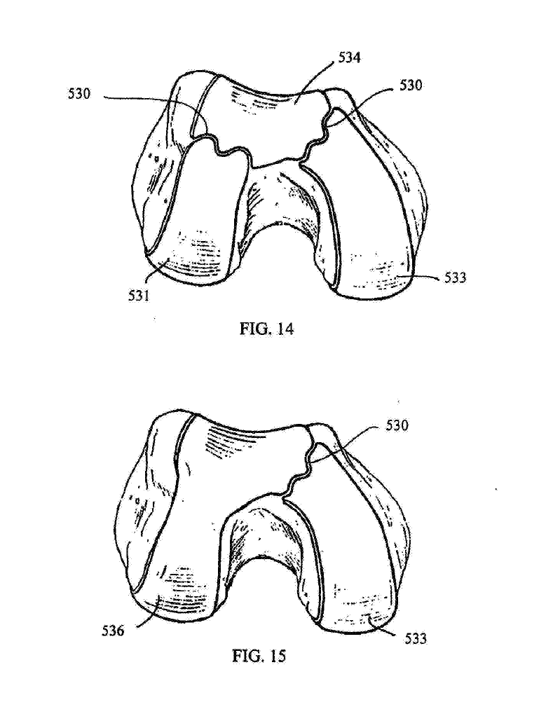

It is beneficial to place all of the implants through small incisions. As seen in FIG. 9, the femoral implants include a first sub-component 131 to resurface the articulating surface of the lateral condyle and a second sub-component 133 to resurface the articulating surface of the medial condyle and a third sub-component 134 to resurface the femoral trochlea. Alternatively, as shown in FIG. 12, the femoral implants are fitted together and unconstrained wherein a first sub-component 431 resurfaces the lateral condyle, a second sub-component 433 resurfaces the medial condyle and a third sub-component 434 resurfaces the femoral trochlea. Optionally, as seen in FIG. 10, the femoral implants may include a first sub-component 133 to resurface the articulating surface of the medial condyle and a second sub-component 136 to resurface the articulating surface of the lateral condyle and the femoral trochlea. Alternatively, as shown in FIG. 13, the femoral implants are fitted together and unconstrained wherein a first sub-component 433 resurfaces the medial condyle and a second sub-component 436 resurfaces the lateral condyle and the femoral trochlea. In an alternate embodiment the interfaces between femoral sub-components are engaged by a meshed structure 530 to provide a uniform transition for patellar articulation on the femoral component between the trochlear sub-component 534 and each condylar sub-component 531 and 533 as shown in FIG. 14. Referring to FIG. 15, a meshed interface 530 may be constructed between a trochlear-condylar sub-component 536 and an adjacent condylar sub-component 533.

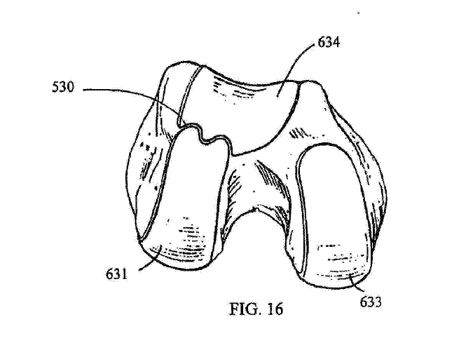

Alternatively, as shown in FIG. 16, a meshed interface 530 may be used for the lateral condylar sub-component 631 to trochlear sub-component 634 transition because of the relatively higher patellofemoral loading along the lateral aspect of the trochlea, and an independent and unconstrained medial condylar sub-component 633 used to resurface the medial condyle. Referring to FIGS. 14, 15 and 16, the meshed interface 530 structure provides engagement between adjacent sub-components that generally limits relative medial to lateral translation of the sub-components one to the other. FIG. 11 is an illustration of an optional femoral condylar sub-component structured as a flexible implant. The outer surface of the condylar implant is a thin sheet of material and the inner surface may be ridged 170.

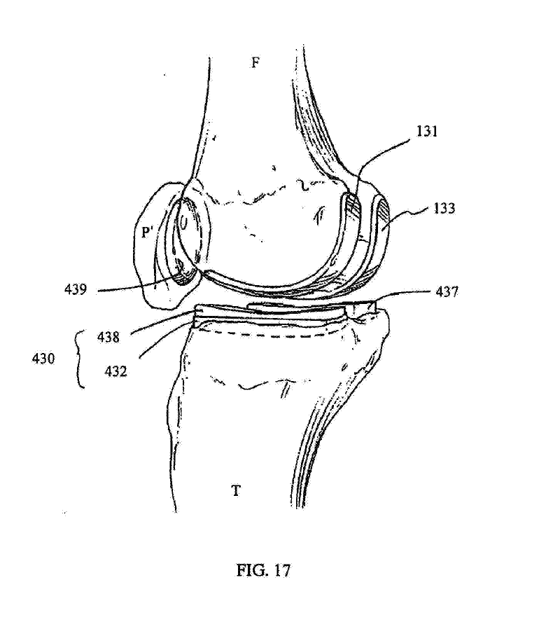

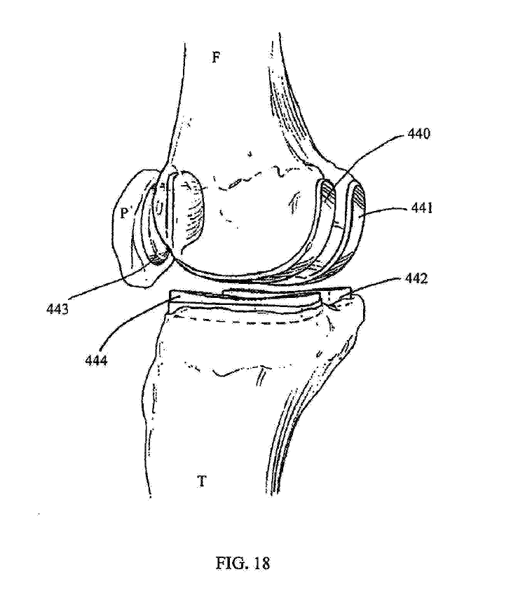

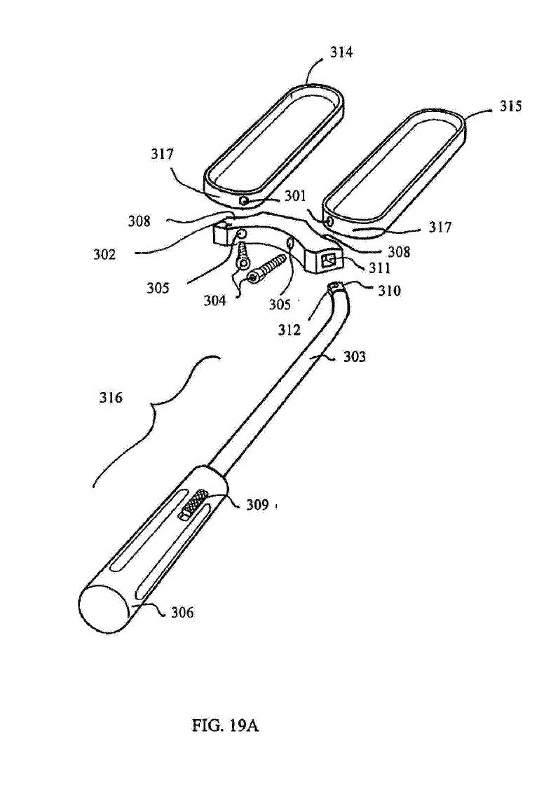

Referring to FIGS. 17 and 18, total knee arthroplasty is comprised of implants that resurface the femoral condyles and trochlea and the tibial articular surfaces per the present invention. In FIG. 17, the femoral F condyles are resurfaced with condylar sub-components medially 133 and laterally 131, the tibial T articular surfaces are resurfaced with tibial sub-components medially 437 and laterally 430. The tibial components comprised of a bearing insert 438 and a baseplate sub-component 432. The patella P is resurfaced with patellar component 439. Optionally as shown in FIG. 17, the femoral trochlea is not resurfaced. In FIG. 18, the femoral condyles are resurfaced with a condylar sub-component of integral structure medially 441. The lateral condylar sub-component 440 and trochlear component are integral, the tibial articular surfaces are resurfaced with tibial sub-components medially 442 and laterally 444. The patella is resurfaced with patellar component 443.

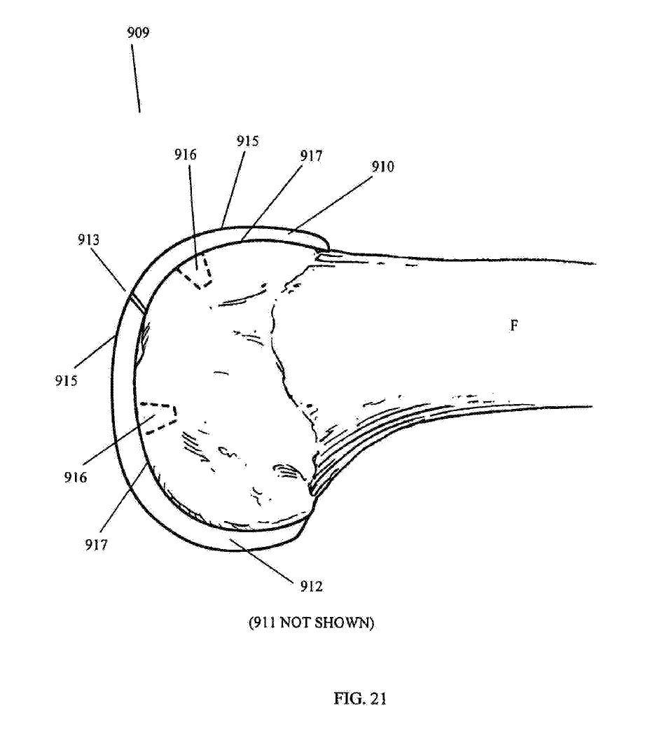

Referring to FIG. 21, the distal femur F is prepared using TGS. The femoral component 909 resurfaces the distal femur F and comprises a plurality of sub-components 910, 911 and 912 each having an inner surface 917 and an opposing articulating surface 915. The inner surface 917 and articulating surface 915 extending between a medial edge and a lateral edge. The inner surface of each sub-component having one or more fixation posts 916. Alternatively, the condylar sub-components having a stabilizing fin (not shown) generally in a sagittal plane along the inner surface 917.

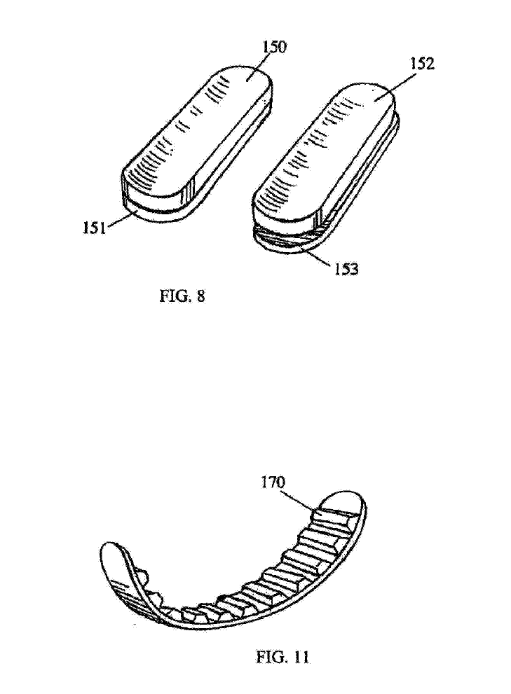

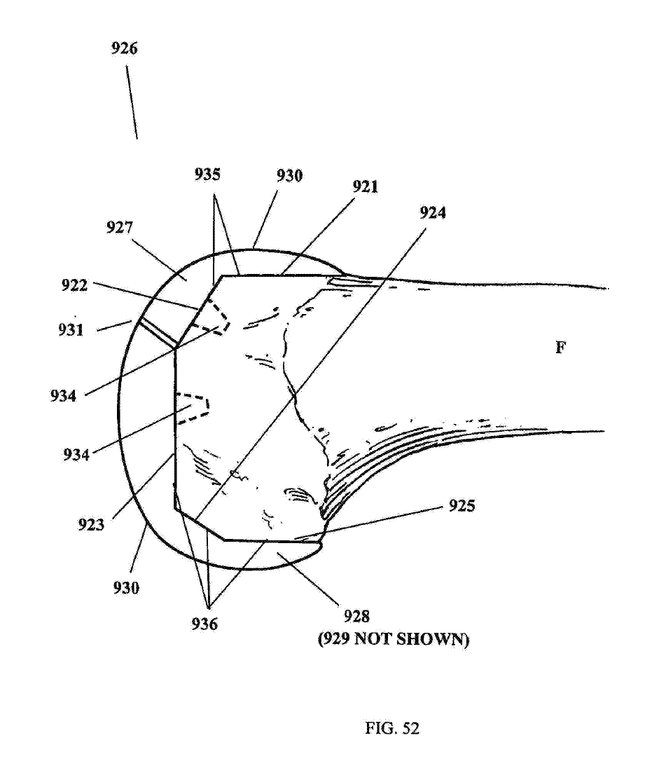

Alternatively, as described earlier and shown in FIG. 52, the distal femur may be prepared with planar resections forming a posterior resection 925, a distal posterior chamfer resection 924, a distal resection 923, a distal anterior chamfer 922 and an anterior resection 921. The femoral component 926 is comprised of a trochlear sub-component 927 with an inner surface 935 structured for attachment to the prepared femoral trochlea and interface 931 structured to engage or join trochlear sub-component 927 to adjacent condylar sub-components 928 and 929. The trochlear sub-component 927 has an outer articular surface 930 on which the patella articulates. In flexion the patellofemoral contact area transitions from the trochlear sub-component 927 to the femoral condylar sub-components 928 and 929 crossing the interface 931. The trochlear sub-component 927 may be structured with one or more posts 934 to provide stability between the implant and supporting bone. The condylar sub-components 928 and 929 have an inner surface 936 structured for attachment to the prepared femoral condyles and interface 931 structured to engage or join the trochlear sub-component 927. The trochlear-condylar sub-component interface is described in detail below. Optionally, the trochlear-condylar sub-component interfaces may be unconstrained or partially constrained or fully constrained when fully assembled. The condylar sub-components may be structured with one or more posts 934 on each sub-component to provide stability between the implant and supporting bone. Alternatively, a fin (not shown) in a generally sagittal plane may be incorporated on the inner surface of the condylar sub-components to provide stability between the implant and supporting bone. For the tibial component, as depicted in FIG. 8, the tibial baseplate sub-components 151 and 153 with corresponding tibial inserts 150 and 152 may be structured as independent tibial baseplates for the medial and lateral compartments.

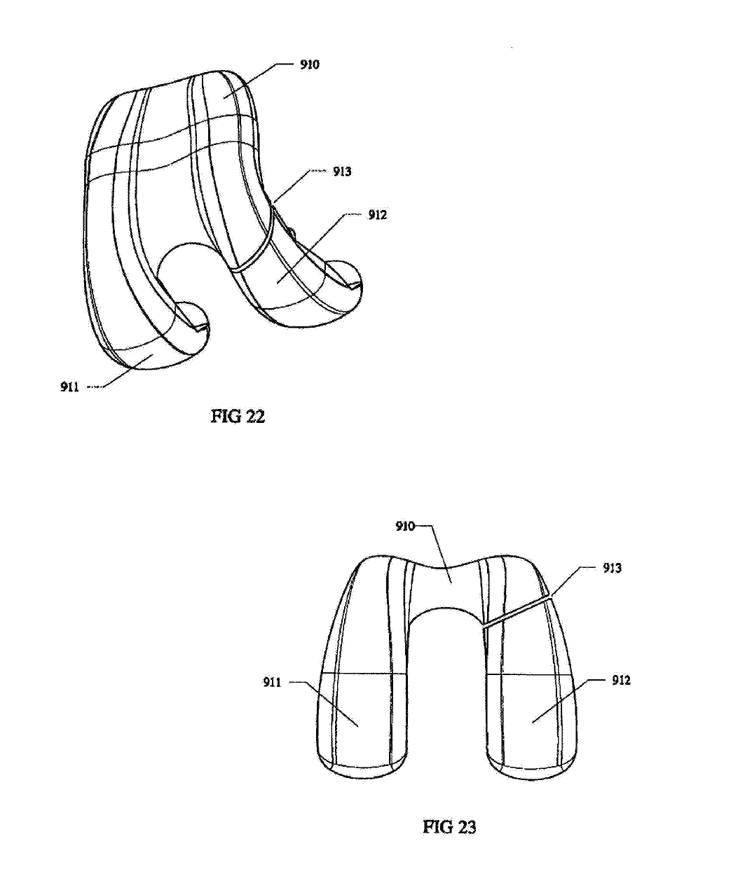

Referring generally to FIGS. 22 through 27, the femoral component of the current invention can be sectioned in various locations to facilitate passage through a small incision and into the joint cavity. Referring to FIGS. 22 and 23, the trochlear sub-component 910 and the lateral condylar sub-component 911 are of unitary construction and the medial condylar sub-component 912 is joined or engaged thereon. The interface 913 between sub-components is unconstrained leaving the sub-components free standing. Alternatively, the interface 913 is partially constrained as described in detail below. In yet another embodiment, the interface 913 is fully constrained when assembled as described in detail below. Alternatively, the trochlear sub-component 910 and medial condylar sub-component 912 are of unitary construction and the lateral condylar sub-component 911 is joined or engaged thereon. The modular interface 913 between the sub-components may be positioned in the “tide mark” region of the distal femoral surface to minimize the effect of the transition on the mating patella or patellar component or tibial component. One embodiment of the present invention is to provide the trochlear sub-component 910 and lateral condylar sub-component 911 as a unitary sub-component to facilitate placement through a small incision medial to the patella and to provide a continuous surface along the lateral aspect of the patellar groove for uniform patellar tracking. In normal knee kinematics the “Q” angle of the quadriceps mechanism pulls the patella laterally on the femoral component. Hence, there are higher contact forces along the lateral aspect of the patellar groove. Alternatively, if the pathology of the knee is less severe it is likely that the lateral femoral condyle is functional and the medial femoral condyle and trochlea are compromised by arthritis. In which case a unitary femoral sub-component to replace the trochlea and medial femoral condyle is applicable.

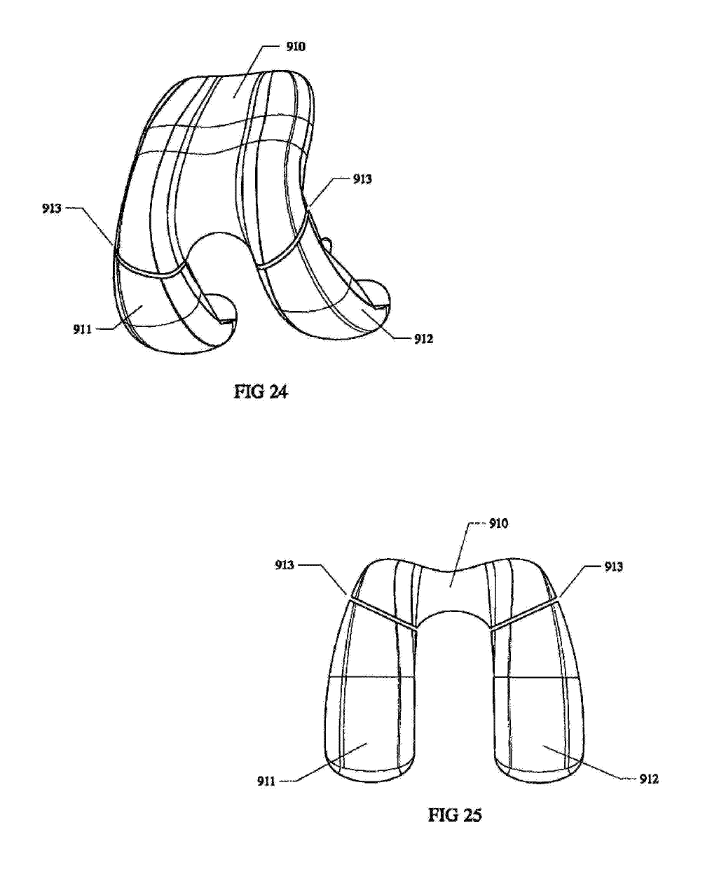

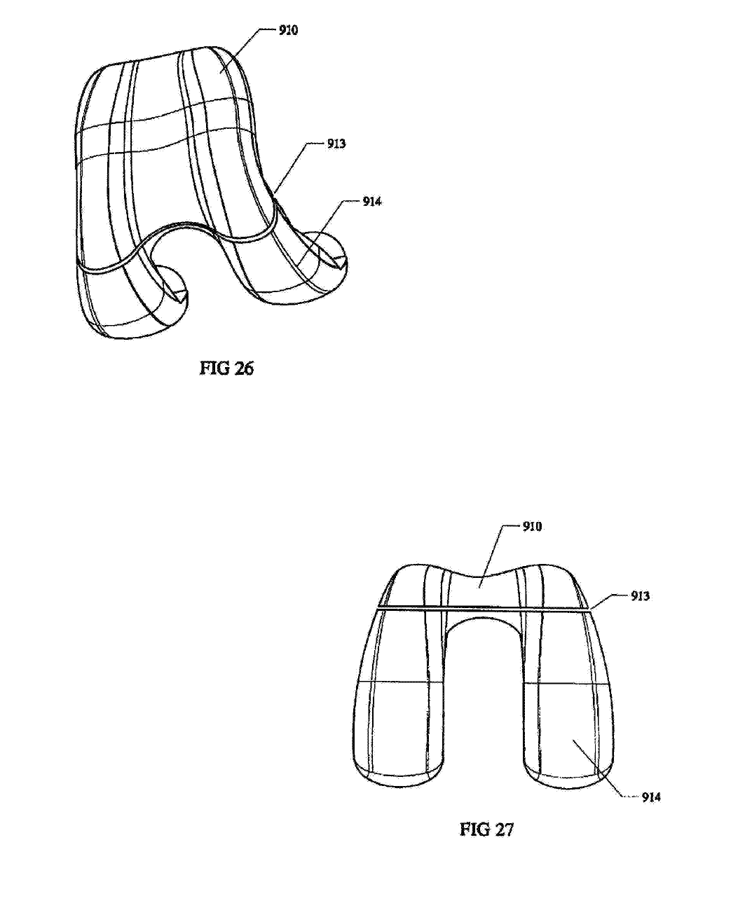

Referring to FIGS. 24 and 25, one embodiment for the femoral component is comprised of three sub-components structured with independent trochlear 910, medial condylar 912 and lateral condylar 911 sub-components with modular interfaces 913 generally in the anterodistal region of the femoral component. The articular surfaces, those surfaces on which mating components slide, provide a contoured surface aligned across the modular interfaces 913 to provide smooth transition of mating components. A sequence for implanting the femoral sub-components is to place the condylar sub-components 911 and 912 first followed by the trochlear sub-component. The trochlear sub-component is passed through the small or minimally invasive incision and joined to the lateral and medial condylar sub-components. The three sub-components are in approximate position on the distal femur when joined and are forced into final position as the components are fully assembled and secured to the femur. As previously described the interfaces 913 between sub-components can be unconstrained or free standing, partially constrained, or fully constrained. Each of these embodiments is described in detail below and all are applicable in each of the femoral component embodiments of the current invention. In yet another femoral component embodiment as shown in FIGS. 26 and 27, an independent trochlear sub-component is joined or engaged with an independent condylar sub-component 914 comprised of a unitary medial and lateral condylar sub-component with the interface 913 between the two sub-components generally in the anteriodistal region of the femoral component.

Looking specifically at the sub-component interface embodiments, as described above the interface, as found between femoral sub-components and between tibial sub-components may be unconstrained, partially constrained or fully constrained when the respective femoral or tibial sub-components are fully assembled. In addition, the interface may be unconstrained or partially constrained during assembly to facilitate assembly within the joint cavity and onto supporting bone surfaces. The engaging mechanism or the joining mechanism may be structured to become more constrained as adjacent sub-components are brought into closer proximity to one another during assembly. Referring to FIGS. 49 A and B, a tapered boss 962, similar to that described above and shown in FIGS. 32 A and B, is structured to allow the condylar sub-components 928 and 929 to angulate generally in a transverse plane. Referring again to FIGS. 49 A and B, the condylar sub-components 928 and 929 angle inward with a gap 963 between adjacent sub-components. Alternatively, the condylar sub-components 928 and 929 may angle outward, or angle in a similar medial to lateral direction relative to the trochlear sub-component 927 to simplify assembly of the femoral sub-components within the confines of the joint cavity. Optionally, threaded fasteners (not shown) are placed in clearance holes 961 to fasten sub-components together.

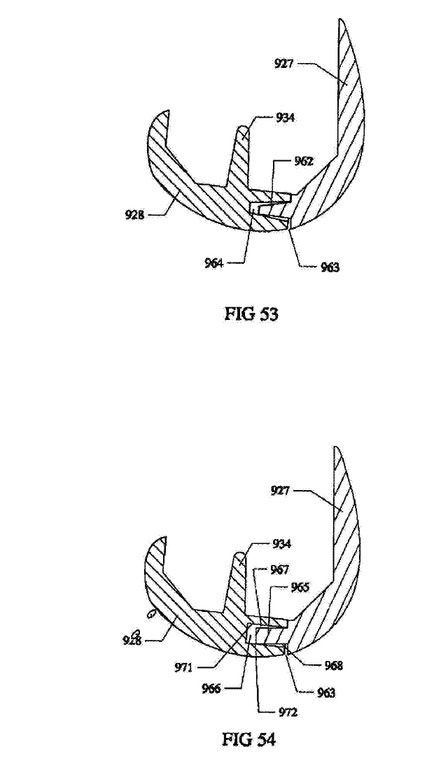

It may be beneficial to allow the condylar sub-components to angulate and translate one to another while assembling them within the confines of the joint cavity. Referring to FIG. 53, which is a cross sectional view of FIGS. 49 A and B, the boss 962 may be structured with a rectangular cross section and inwardly tapered opposing sides. The receiving pocket 964 structured to snuggly receive the boss 962 when fully assembled, but provide an unconstrained interface between the trochlear sub-component 927 and the condylar sub-component 928 as the sub-components are initially placed together for assemble within the confines of the joint cavity. Hence, the trochlear sub-component may be angulated and translated relative to one or both of the condylar sub-components by the surgeon to facilitate assembly. Alternatively, as shown in FIG. 54, the boss 965 on the trochlear sub-component 927 may have a rectangular cross section and parallel opposing sides and structured to fit loosely within a receiving pocket 966 in the condylar sub-component 928 for an unconstrained interface during assembly and unconstrained or partially constrained when fully assembled. The fully assembled trochlear sub-component to condylar sub-component interface is unconstrained when a gap 963 remains between the sub-components after assembly onto the supporting bone. Alternatively, the trochlear sub-component to condylar sub-component interface is partially constrained when the gap 963 is closed between the sub-components after assembly onto the supporting bone. In this case, the adjacent sub-components are able to translate in the plane of the interface. Optionally, the superior 967 and inferior 968 surfaces of the boss 965 may be structured to snuggly slide within opposing superior 971 and inferior 972 surfaces of the receiving pocket 966 to provide a partially constrained engaging interface mechanism preventing superior-inferior relative translation and angulation between the adjacent sub-components. Alternatively, the vertical side surfaces of the boss 965 may be structured to snuggly slide within opposing vertical side surfaces of the receiving pocket 966 to provide a partially constrained engaging interface mechanism preventing mediolateral relative translation and angulation between adjacent sub-components. Ultimately, each femoral sub-component 927, 928 and 929 is secured to its supporting bone by bonding with bone cement or by bone ingrowth.

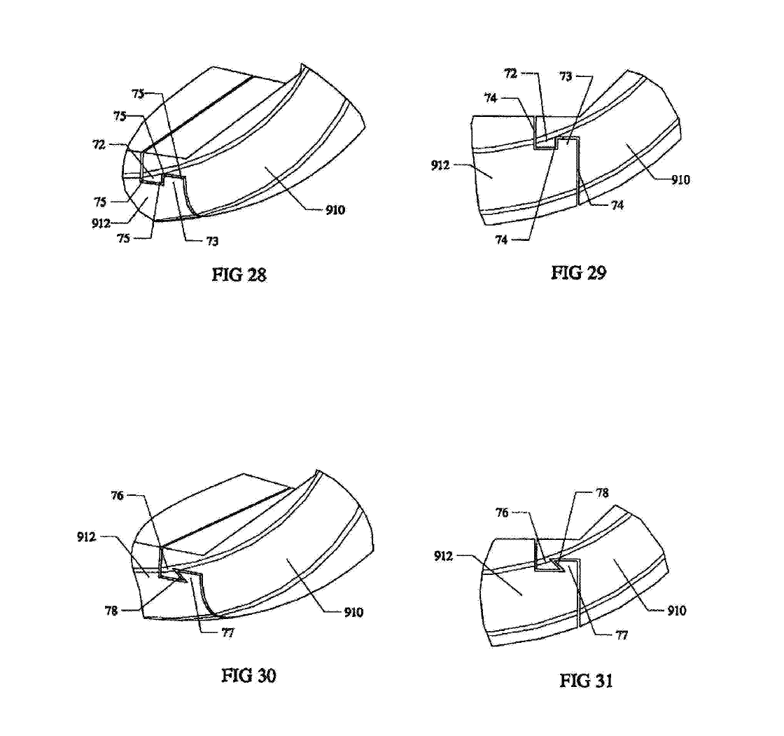

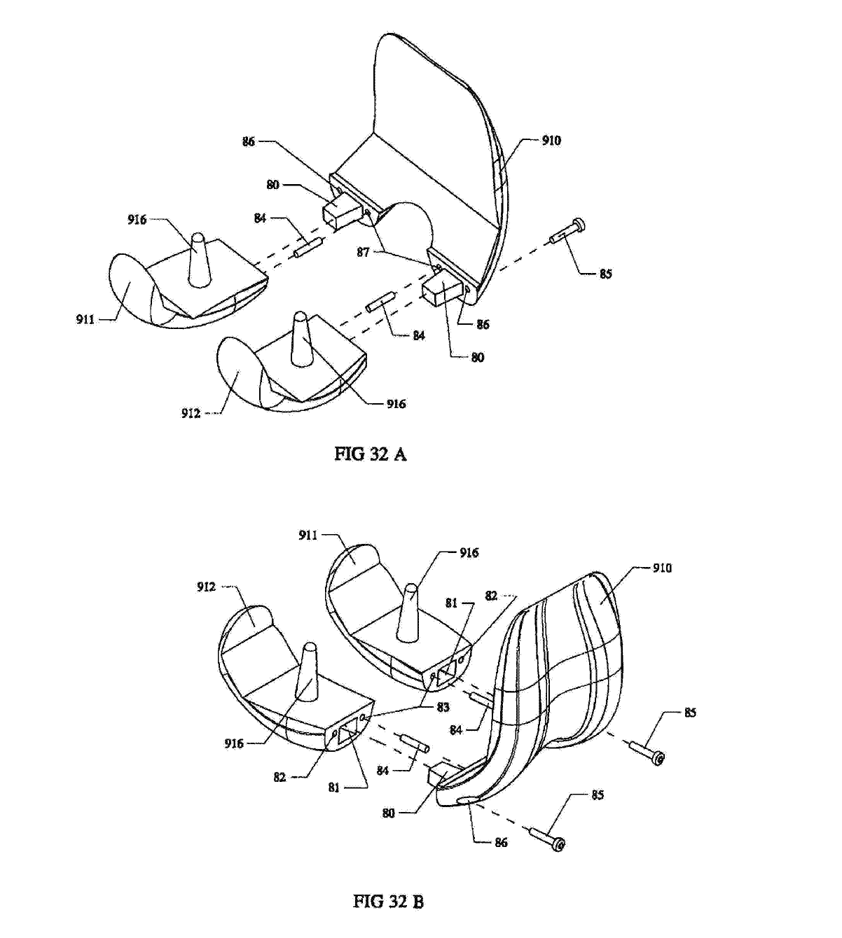

In tri-compartmental knee replacement it is beneficial to recreate normal kinematics. To accomplish the alignment and orientation of each femoral sub-component is optimized to maintain proper ligament tension and balance throughout a full range of motion of the knee. Hence, the alignment and orientation of each sub-component to adjacent sub-components and of the femoral component to the tibial and patellar components are critical. As shown in FIGS. 28 and 29, the interlock between the trochlear sub-component 910 and condylar sub-components 911 and 912 is with interlocking bosses 72 and 73. The axial clearance 74 between the sub-components is structured to allow moderate angulation generally in a sagittal plane and constrained axial translation and constrained angulation in a transverse plane. Optionally, the axial clearance 74 can be increased to allow greater axial translation and angulation generally in a sagittal plane. In addition, placing a radius on the corners 75 of the two bosses 72 and 73 and in the opposing corners increases angulation generally in a sagittal plane. Before securing the implants to supporting bone, axial rotation and orthogonal translation are unconstrained this is beneficial in assembling the sub-components within the joint cavity. Once secured to bone, the condylar sub-component boss 73 traps the trochlear sub-component boss against supporting bone. Alternatively, the trochlear boss 72 may be placed distal to the condylar sub-component boss in which case it would trap the condylar sub-component boss. Optionally, as shown in FIGS. 30 and 31, orthogonal translation generally in a superior-inferior direction can be constrained by the addition of a partial dovetail 78 to the condylar sub-component boss 76 and the trochlear sub-component boss 77. Orthogonal translation generally in a mediolateral direction remains unconstrained and facilitates placing the trochlear sub-component onto the medial and lateral condylar sub-components from the medial or lateral aspect of the femur. Such assembly of the trochlear sub-component to the condylar sub-components may be beneficial when the condylar sub-components are independently secured to the prepared femoral condyles as described above followed by placement of the trochlear sub-component due to the ability to slide the trochlear sub-component between the patella and femur while engaging the interlocking bosses 76 and 77.

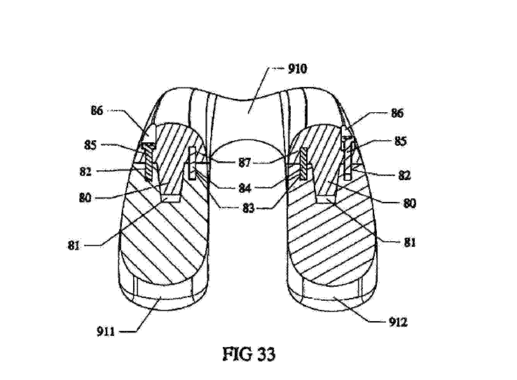

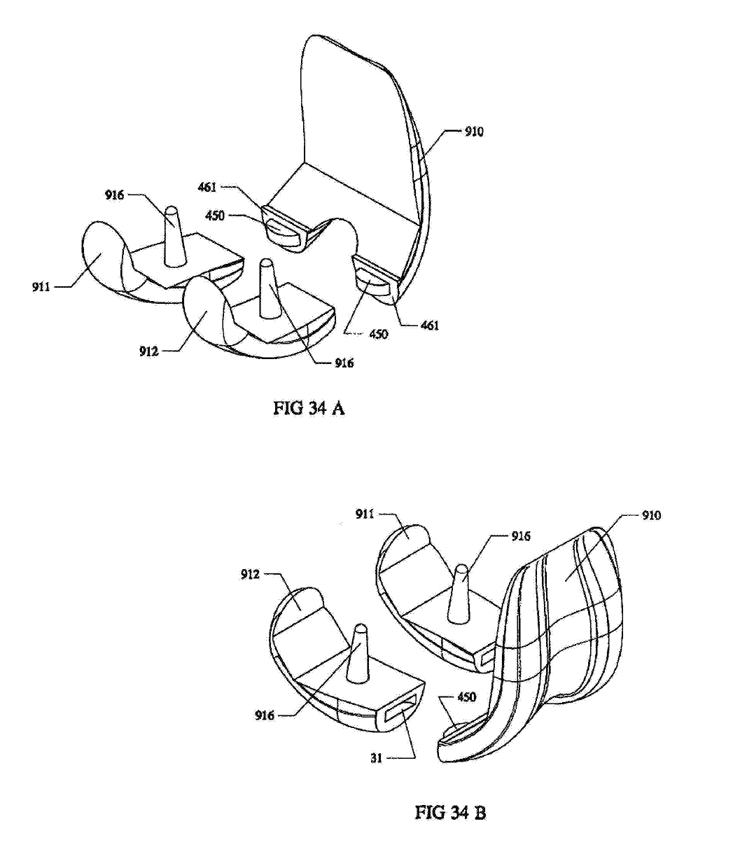

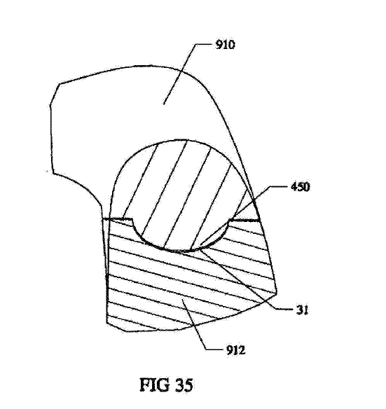

Referring to FIGS. 34 and 35, optionally, orthogonal translation generally in a sagittal plane and axial rotation may be constrained by capturing a boss 450 of rectangular cross section and protruding away from the trochlear sub-component 910 within a receiving pocket 31 of matching shape and rectangular cross section formed in the condylar sub-component 911 or 912. Alternatively, the boss may be on the condylar sub-component 911 or 912 and the pocket in the trochlear sub-component 910. In either case, a relatively short boss is needed to facilitate assembly within the joint capsule. Alternately, as shown in FIGS. 32 and 33, the boss 80 of the trochlear sub-component 910 is tapered in a sagittal cross section and the taper of the corresponding pocket 81 in the condylar sub-component 911 or 912 is tapered to snugly receive the trochlear sub-component boss 80 allowing less constraint in angulation generally in a sagittal plane as the adjoining sub-components are fitted together, which would facilitate assembly within the joint capsule and provide a constrained interface when the taper junction is fully seated. Optionally, the boss may also be tapered in a transverse plan to provide unconstrained angulation generally in a transverse plan to facilitate assembly within the joint cavity. As the boss 80 and pocket 81 are seated, this interface becomes increasingly constrained to a full constraint when fully seated. Alternatively, the boss 80 and receiving pocket 81 may be of matching circular, oval or other suitable cross section structured with or without tapers and with the pocket structured to snugly receive the boss.

To simplify assembly and increase stability of the interface a dowel pin 84 is pressed into the trochlear sub-component receiving hole 87 to be received by a mating hole 83 in the condylar sub-component. Optionally, the trochlear sub-component may be structured with a clearance hole 86 to accommodate a threaded fastener 85 that threads into a threaded receiving hole 82 and provides a means to apply a compressive retaining force across the sub-component interface. To avoid disrupting the articular surface of the trochlear sub-component, the clearance hole 86 is positioned to be either medial or lateral to the articular path of the patellar component or of the tibial bearing component. Fasteners may include, but are not limited to, the interference of the tapered elements, screws and threaded fasteners, expanding pins or bars, press fit pins or bars, other fastener means, or a combination of these.

Referring again to FIG. 32, alternatively, the boss 80 may be structured to be flexible generally in a sagittal plane by relieving the superior and inferior surfaces of the tapered element at its base. Such a flexible interconnection between adjoining sub-components may be advantageous in accommodating regional variations in the kinematically prepared support surfaces of the distal femur.

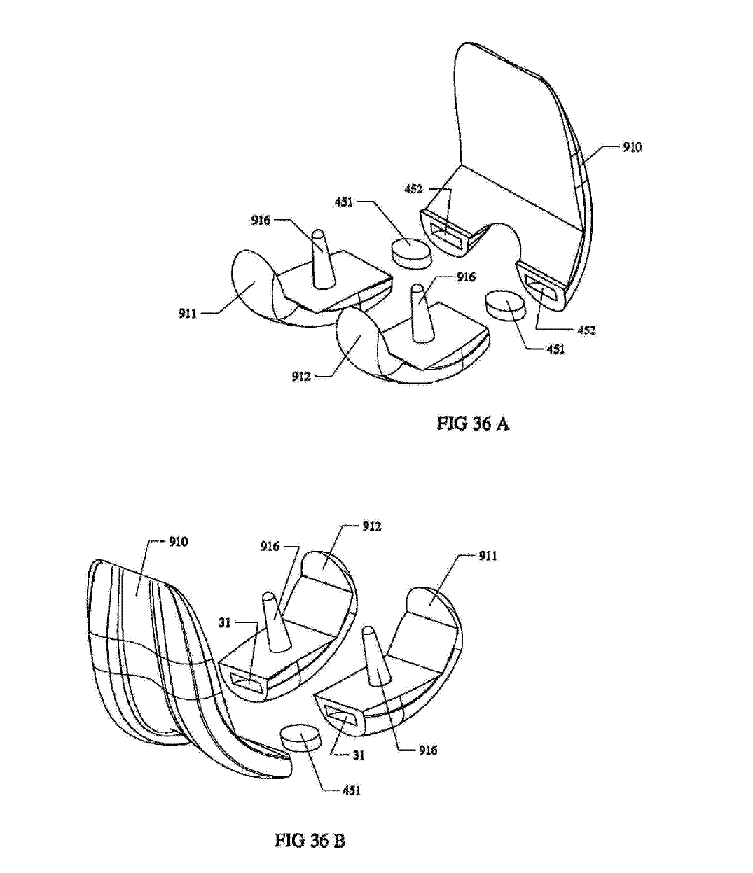

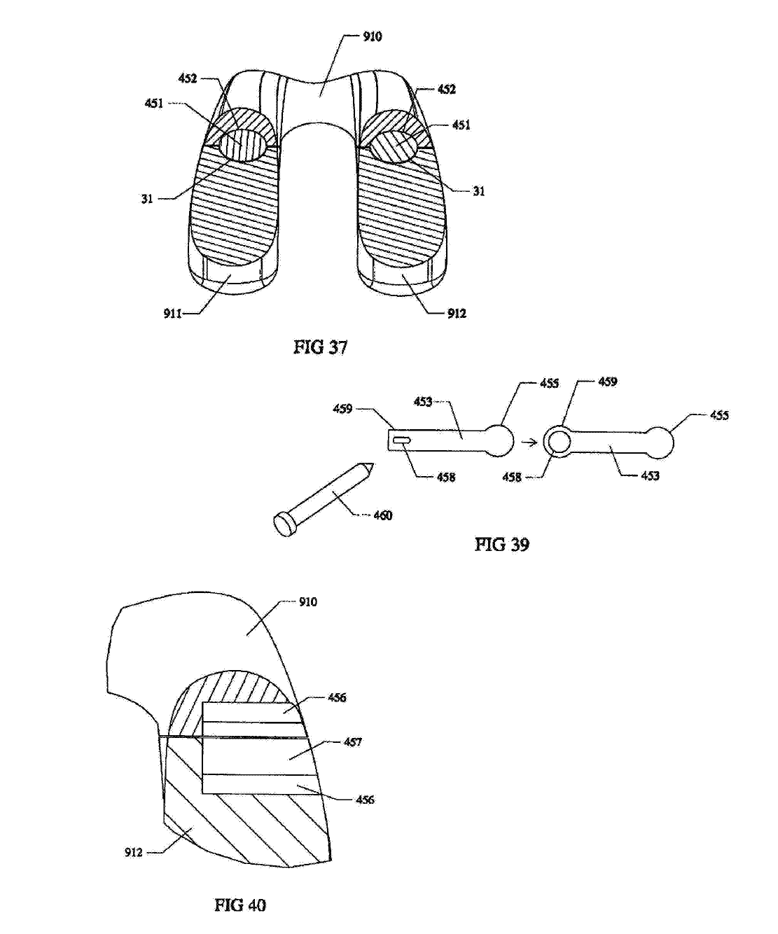

As described above, it may be advantageous to have a flexible interconnection between adjoining sub-components. Referring to FIGS. 36 and 37, an alignment tab 451 is flexible and is interposed between the trochlear sub-component and adjoining condylar sub-components 911 and 912. The alignment tab 451 is made of a flexible material, such as polyethylene, urethane or other suitable plastic material; or a metal such as NP35N, stainless steel, Nitinol or other suitable metal that is structured to be flexible. The alignment tab 451 is cylindrical. Alternatively, the alignment tab 451 may be oval, rectangular, or of any suitable shape and cross section. The receiving pocket 31 in the condylar sub-components 911 and 912 and receiving pocket 452 in the trochlear sub-component are structured to match the shape and cross section of the alignment tab 451 to provide a stable sliding interface between the alignment tab and the sub-components. Alternatively, the alignment tab 451 may be tapered inwardly as it protrudes towards the condylar sub-components or the trochlear sub-component, and the receiving pockets 31 and 452 structured to match such tapers providing a snug fit between the alignment tab and mating condylar sub-components and the mating trochlear sub-component.

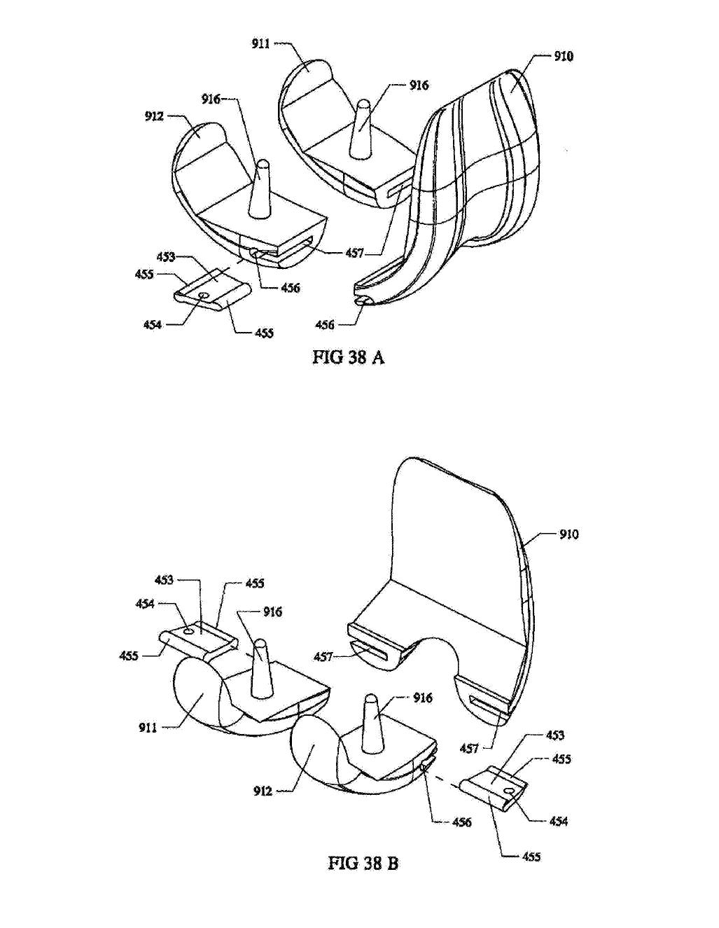

It may be beneficial for the alignment tab to be temporarily placed into the sub-components to simplify assembly and attachment to supporting bone within the joint capsule. Referring to FIGS. 38, 39 and 40, first, bone cement is placed on the inner surfaces of the sub-components and on the prepared surfaces of the distal femur. The condylar sub-components 911 and 912 and trochlear sub-component 910 are placed into the joint cavity and onto the supporting bone. The sub-components are then assembled using a flexible alignment tabs 453 placed into mating slots 457 in the trochlear sub-component and the condylar sub-components. Two alignment tabs 453 are required, one for the medial condylar sub-component 912 attachment to the trochlear sub-component 910 which is placed from the medial side and one for the lateral condylar sub-component 911 attachment to the trochlear sub-component 910 which is placed from the lateral side. The condylar sub-components are impacted with the knee in flexion followed by impaction of the trochlear sub-component with the knee in extension. Excess bone cement is removed and the cement allowed to cure. Trial tibial implants and trial patellar implants may be placed to provide compressive loading of the femoral sub-components while the bone cement cures. In one embodiment as shown in FIG. 38, the alignment tab 453 has cylindrical edges 455 structured to slide into slots 457 in the condylar sub-components and trochlear sub-component configure to match the shape and cross section of the alignment tab 453. The alignment tab cylindrical edges 455 are structured to engage the cylindrical recesses 456 in the condylar sub-components and the trochlear sub-component.

Alternatively, one of the cylindrical edges of the alignment tab 453 may be structured to collapse and expand to simplify assembly of the sub-components within the joint cavity. Referring to FIG. 39, the expandable edge 459 of the alignment tab is structured with a slot 458 running the length of the alignment tab. The cylindrical edge 455 of the alignment tab 453 is placed into the receiving slot 457 of either the trochlear sub-component or one of the condylar sub-components, then slid into the receiving slot 457 of the mating sub-component. An expansion pin 460 is placed into the slot 458 to expand the expandable edge 459 to engage the cylindrical recess 456 in the mating sub-component. This is repeated for the other condylar sub-component and the femoral component is secured to the prepared femur as described above. After the bone cement has sufficiently cured, the alignment tabs 453 are removed by hooking the removal hole 454. Alternatively, a suture may be tied to the removal hole to facilitate easy removal of the alignment tabs. Alternatively, the alignment tabs 453 may be placed into the receiving slots 457 using tether devices as described in U.S. patent application Ser. No. 11/186,485.

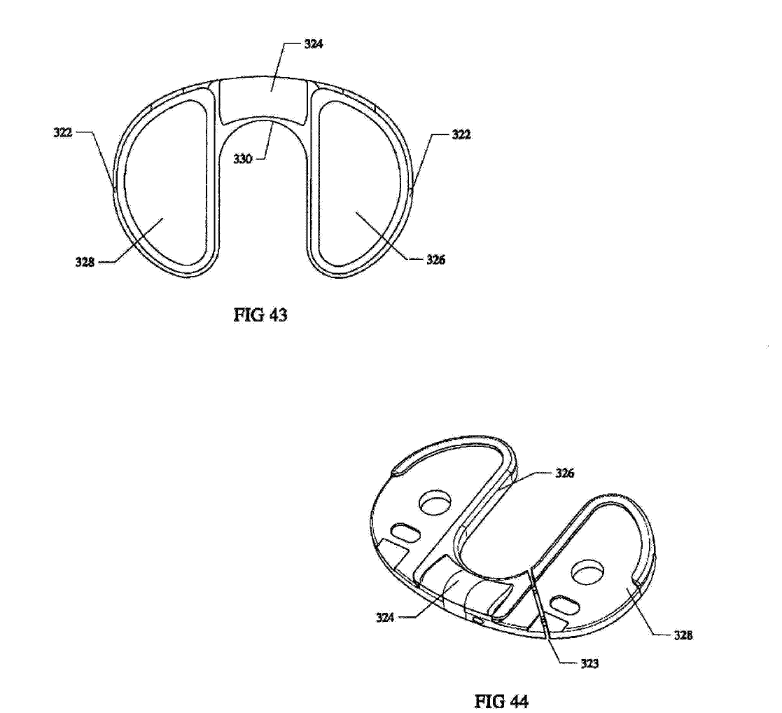

Turning to the tibial implants, as described above the tibial baseplate component may be unitary in construction as shown in FIG. 43, to cover the prepared surface of the tibial plateau as relates to the knee. The medial baseplate 328 and lateral baseplate 326 may be symmetrical to allow use of one design for right or left knees. Alternatively, the medial baseplate 328 and lateral base 326 may be asymmetric requiring left and right designs. The bridge 324 between the medial 328 and lateral 326 baseplates is shown with a narrow anterior to posterior dimension to enable placement of the bridge 324 anterior to the insertion of the anterior cruciate ligament to preserve supporting bone in an anterior cruciate sparing total knee design. Optionally, the posterior surface of the bridge 330 may be moved posteriorly (not shown) for an anterior cruciate sacrificing total knee design. Optionally, the posterior surface of the bridge may be moved further posteriorly (not shown) for a cruciate sacrificing (anterior and posterior cruciate ligaments) total knee design, commonly known as a posterior stabilized total knee. The proximal surfaces of the medial 328 and lateral 326 baseplates are recessed with a shoulder 322 around the circumference of the recess providing one form of capture mechanism or restraint for a tibial bearing insert (not shown). Other tibial bearing insert to baseplate locking means are known in the art and include dovetail mechanism, locking tabs, locking keys and pins and other fasteners to secure a tibial bearing insert onto a baseplate.

If structured as a unitary component, the tibial baseplate provides a capture mechanism for a fixed bearing or a mobile bearing insert for either the medial or lateral tibiofemoral compartment. As an option, a single platform is structured to provide a fixed bearing capture mechanism for the medial tibiofemoral compartment and a mobile bearing capture mechanism or a simple platform to receive a mobile bearing insert for the lateral tibiofemoral compartment. Since right and left tibial baseplates are required, the same baseplate may be used for a mobile bearing medial insert and a fixed bearing lateral insert.

As shown in FIG. 44, the tibial baseplate is optionally structured as a two piece component wherein the sub-components are joined within the confines of the joint cavity. Tibial inserts 438 and 445 are structured to engage the tibial baseplates 326 and 328. The split 323 between the medial baseplate 328 and lateral baseplate 326 may be medial of the bridge 324; however the split 323 may be located anywhere along the bridge and angle medially or laterally with respect to the sagittal plane, or be parallel to it. The benefit of placing the split 323 medially and angled is three fold, first this provides additional cross sectional area for an interconnect mechanism, second it provides easy access perpendicular to the split 323 via the medial parapatellar incision for fastener placement, and third it provides an extension onto which an inserter can be attached to facilitate placement of the lateral tibial baseplate sub-component 326 through a medial parapatellar incision. Alternatively, the interconnection between the medial baseplate sub-component 328 and the lateral baseplate sub-component 326 at split 323 is fully constrained to hold the medial 328 and lateral 326 sub-components in a common plane and to hold the divergence of the sub-components at a fixed angle. Optionally, the interconnection at split 323 is partially constrained.

As in the femoral sub-components, the tibial baseplate may be structured as a unitary piece, or as a plurality of components. In the later case, the interface between tibial baseplate sub-components may be unconstrained, partially constrained or fully constrained. The sub-component interface embodiments described for the femoral sub-components are applicable to joining or engaging the tibial sub-components and this is implied by reference. In addition, the sub-component interface embodiments described for the tibial baseplate sub-components are applicable to joining or engaging the femoral sub-components where they may differ from those described above. The tibial baseplate sub-components are manufactured from a suitable metal, to include cobalt chromium alloy, titanium or titanium alloy or stainless steel; or from zirconia or alumina ceramic. The sub-components may be machined or cast or molded. Manufacturing methods include machining, wire and plunge EDM, and other suitable fabrication process.

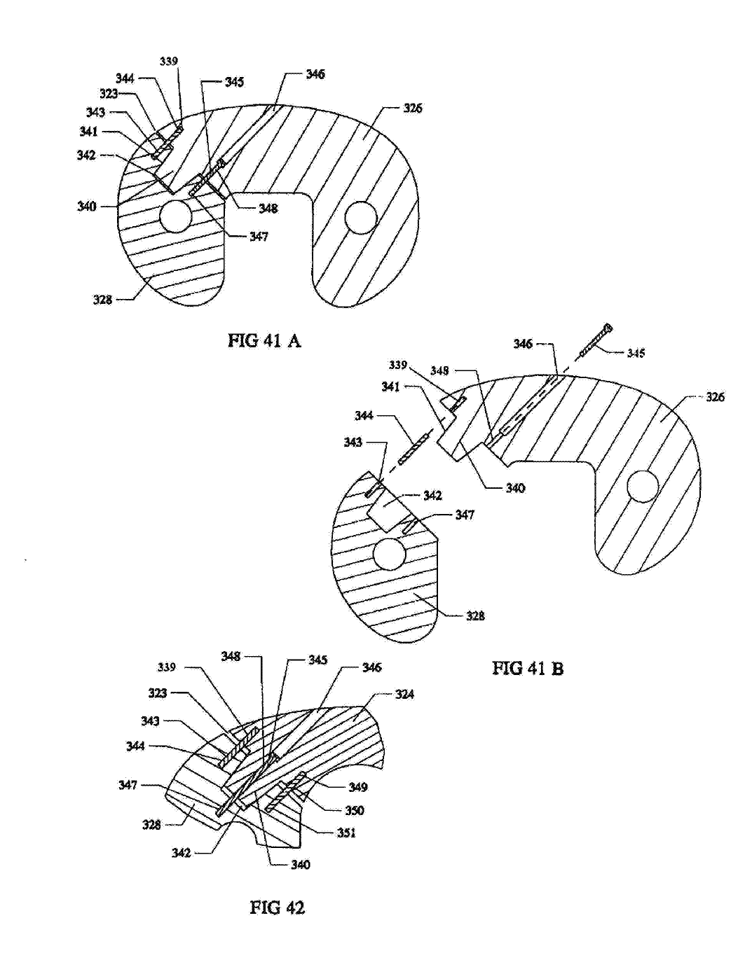

Referring to FIGS. 41 and 42, in an alternate embodiment the tibial baseplate is sectioned along one of the sides of the opening for the tibial eminence with such interface between sub-components angling away from a sagittal plane passing through the center of the knee. In an alternate embodiment the interface between sub-components is towards the medial condyle to position the interface below the surgical incision and to the side of the patellar ligament. As shown in FIG. 41, a boss 340 extends from and the bridge 324. The boss 340 may be rectangular in cross section. The inferior-superior dimension of the boss 340 being less than that of the corresponding inferior-superior dimension of the tibial baseplate sub-components 326 and 328 in the region of the bridge 324. The sub-component interface may be structured for relatively constrained assembly by structuring the boss 340 to have parallel surfaces on opposing sides of the boss protruding from the interface surface of the lateral sub-component 326. The receiving pocket 342 is structured with a shape and cross section to slidably fit the mating boss 340. However, assembly within the joint cavity may be simplified by tapering the boss 340 to allow angulation between the sub-components during assembly and a constrained interface after the sub-components are fully seated. Optionally, as shown in FIGS. 51 A & B, the boss 340 may have parallel surfaces on the superior and inferior surfaces and inwardly tapering surfaces on the vertical surfaces 341 to provide constraint in superior-inferior angulation between the sub-components and minimal constraint to angulation within the plane of the baseplate during assembly. In an alternate embodiment, the interlock between sub-components may include a dowel pin 344 and a threaded fastener 345 as shown in FIG. 41, or may not as shown in FIGS. 51 A & B. Referring again to FIGS. 51 A & B, the baseplate sub-components may be positioned with the boss 340 partially engaged in the receiving pocket 342 (see FIG. 41) enabling the sub-components to be angulated one to the other generally in a transverse plane to orient the sub-components relative to the geometry of the supporting bone of the tibial plateau.

It may be beneficial to allow the baseplate sub-components to angulate and translate one to another while assembling them within the confines of the joint cavity. Referring to FIGS. 51 A and B, the boss 340 may be structured with a rectangular cross section and inwardly tapered opposing sides. The receiving pocket 342 (see FIG. 41) is structured to snuggly receive the boss 340 when fully assembled, but provide an unconstrained interface between adjacent sub-component 326 and 328 as the sub-components are initially placed together for assemble within the confines of the joint cavity. Hence, the baseplate sub-component may be angulated and translated relative one to the other by the surgeon to facilitate assembly. Alternatively the boss 340 may have a rectangular cross section and parallel opposing sides and structured to fit loosely within a receiving pocket 342 for an unconstrained interface during assembly and unconstrained or partially constrained when fully assembled, the boss 340 and receiving pocket 342 being of similar structure as that described above for the femoral sub-components as relating to FIG. 54. The fully assembled baseplate sub-component to sub-component interface is unconstrained when a gap 323 remains between the sub-components after assembly onto the supporting bone. Alternatively, the baseplate sub-component to sub-component interface is partially constrained when the gap 323 is closed between the sub-components after assembly onto the supporting bone. In this case, the adjacent sub-components are able to translate in the plane of the interface. Optionally, the superior and inferior surfaces of the boss 340 may be structured to snuggly slide within opposing superior and inferior surfaces of the receiving pocket 342 to provide a partially constrained engaging interface mechanism preventing superior-inferior relative translation and angulation between the adjacent sub-components. Alternatively, the vertical side surfaces of the boss 340 may be structured to snuggly slide within opposing vertical side surfaces of the receiving pocket 342 to provide a partially constrained engaging interface mechanism preventing mediolateral relative translation and angulation between adjacent sub-components. Ultimately, the baseplate sub-components 328 and 326 are secured to supporting bone by bonding with bone cement or by bone ingrowth.

Optionally, the boss 340 may have inwardly tapering surfaces on the superior and inferior surfaces (not shown) and the vertical surfaces 341 to provide minimal constraint to angulation in any direction between the sub-components during assembly within the joint cavity. In both embodiments the receiving pocket 342 is structured with a shape and cross section to snuggly fit the mating boss 340 thereby provided a fully constrained interface when the sub-components are fully seated. Alternatively, the boss 340 may be structured as a cylindrical or truncated cone or other suitable shape and cross section for engaging or joining the sub-components and the receiving pocket 342 is structured with a shape and cross section to snuggly fit the mating boss 340. Alternatively there may be multiple bosses (not shown) protruding from the interface surface of the lateral baseplate sub-component with receiving pockets structured with a shape and cross section to snuggly fit the mating bosses in the other sub-component. Alternatively, the boss or bosses may protrude from the medial baseplate sub-component with the receiving pockets in the lateral baseplate sub-component.

Referring to FIG. 41, a dowel pin 344 may be pressed fit into a receiving hole 339 in the lateral baseplate sub-component 326. The receiving hole 343 for the dowel pin 344 in the medial baseplate sub-component provides a slip fit for ease of assembly. Alternatively, the dowel pin 344 may be press fit into the medial baseplate sub-component and slip fit into the lateral baseplate sub-component. It may be beneficial to provide a compression force to fully seat the tapered interfaces and to provide a mechanical locking of the sub-components to one another. In one embodiment a threaded fastener 345 is placed through a receiving hole 348 in the lateral baseplate sub-component and treads into a threaded receiving hole in the medial baseplate sub-component. The anterior opening of the clearance hole 346 is enlarged to provide a countersink for the head of threaded fastener 345. Referring to FIG. 42, the threaded fastener 345, clearance holes 348 and 346, and threaded receiving hole 347 may be structured to pass through the boss 340 and receiving pocket 342 allowing for a second dowel pin 349 to be press fit into a receiving hole 350 in the lateral baseplate sub-component thereby providing additional stability to the interface when placed in a receiving slip fit hole 351 in the medial baseplate sub-component.

As described above, there may be patient indications wherein the use of a post attached to the tibial baseplate and extending into the tibial medullary canal is needed to provide additional stability to the implant. Similarly, at times such indications exist for the femoral component wherein the use of a post attached to the femoral component or sub-components and extending into the femoral medullary canal is needed to provide additional stability to the implant. Conventional tibial and femoral knee implants structured for use with modular posts are structured for assembly outside of the joint cavity. Such designs are problematic in less and minimally invasive knee arthroplasty because the limited surgical exposure does not allow sufficient access to place the assembled components into the joint cavity. In the present invention it has been found that the limited surgical exposure allows sufficient access to place a stem into the tibial medullar canal. Similarly for the femoral side, it has been found that the limited surgical exposure allows sufficient access to place a stem into the femoral medullar canal. Hence, in one embodiment of the present invention a stem is passed into the joint cavity and into a prepared hole in the tibial plateau extending to the medullary canal. After which tibial components or sub-components of the present invention as described above are placed into the joint cavity and assembled to the stem. Similarly for the femoral component, in one embodiment of the present invention a stem is passed into the joint cavity and into a prepared hole in the distal femur extending to the medullary canal. After which femoral components or sub-components of the present invention as described above are placed into the joint cavity and assembled to the stem. In one embodiment of the present invention the femoral stem is placed first, followed by the tibial stem, followed by the femoral sub-components, and finally by the tibial sub-components. Alternatively, the femoral stem is placed first, followed by the femoral sub-components, followed by the tibial stem, and finally by the tibial sub-components.

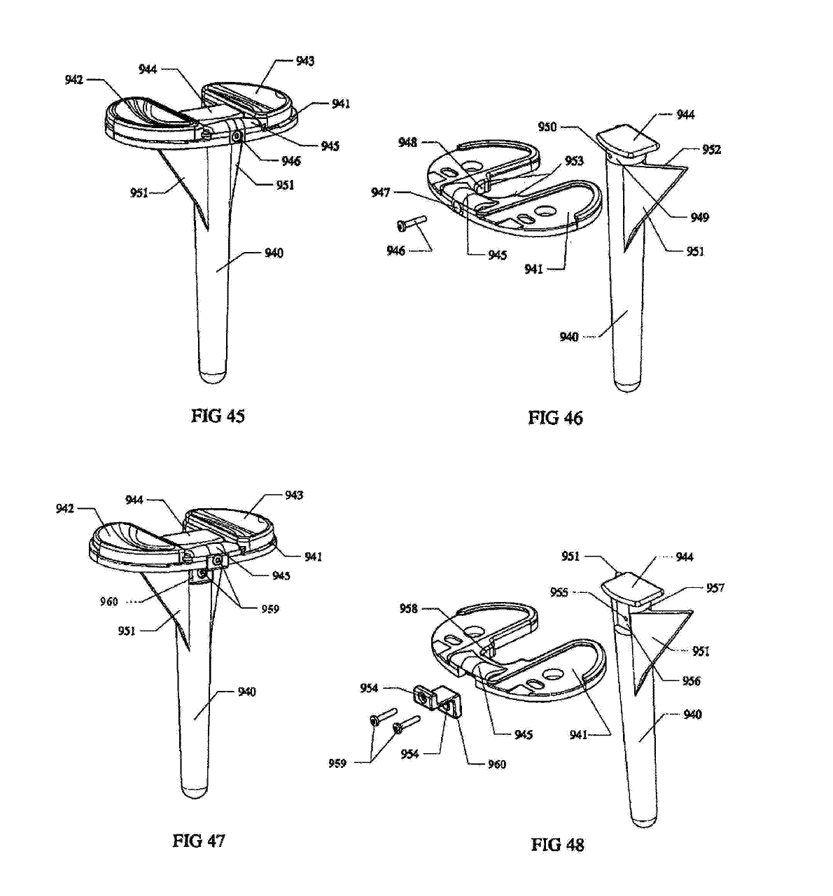

Generally referring to FIGS. 45 and 46, in an alternate embodiment of the invention the tibial component is comprised of a stem sub-component 940 and a unitary baseplate sub-component 941 with bridge 945 and structured to support tibial inserts 942 and 943. Alternatively, medial and lateral baseplate sub-components as described above may be used with the stem sub-component 940, wherein the stem sub-component 940 is placed into the tibia, followed by the medial baseplate sub-component, then the lateral baseplate sub-component. The tibial sub-components are then assembled within the joint cavity. Alternatively, the lateral baseplate sub-component may be placed before the medial baseplate sub-component.

Flexing the knee to greater than 90.degree. provides access to prepare a receiving hole in the proximal tibia for the stem sub-component. A tibial template or trial and a punch commonly know to those skilled in the art are used to prepare the receiving hole. Referring to FIG. 46, the stem sub-component 940 is placed into the tibia with the knee similarly flexed. It may be beneficial to leave the stem approximately 2 mm to 6 mm short of its fully seated position to facilitate placement of the implants with bone cement as will be explained below. If bone cement is to be used, the bone cement is applied to the underside of the baseplate sub-component and onto the tibial plateau. With the knee in extension, the baseplate sub-component 941 is placed into the joint cavity by placing the lateral aspect of the baseplate sub-component 941 through the incision medial to the patellar ligament and above the stem sub-component 940. The baseplate sub-component 941 is then rotated to align with the tibial plateau and is pulled anteriorly until receiving tabs 953 clear the stem capture plate 944. The baseplate sub-component 941 is then brought down to the level of the receiving tabs 953, which in the case of a cemented component have been positioned slightly above the tibial plateau to facilitate placing the baseplate sub-component 941 onto the stem without disrupting the bone cement previously placed on the baseplate sub-component and on the tibial plateau. The baseplate sub-component 941 is pushed posteriorly to slidably engage a receiving groove 949 in the proximal stem sub-component with receiving channel 948 and secured to the stem sub-component 940 with a threaded fastener 946 placed through receiving clearance hole 947 in the baseplate sub-component 941 and threaded into threaded receiving hole 950 in the stem sub-component. Alternatively, other fastening means known in the art may be used, for example cross pins, snap fits, tapered fits or other suitable attachable means. Alternatively, the capture plate 944 may be modular allowing the baseplate sub-component 941 to be placed onto the stem sub-component 940 by lowering the baseplate sub-component 941 onto a receiving post followed by placing the capture plate 944 and securing the capture plate 944 with one or more threaded fasteners place through the capture plate and into the stem sub-component 940. After securing the baseplate sub-component to the stem sub-component the knee is flexed to greater than 90.degree. to provide access for an impaction tool and the tibial component is impacted onto the tibial plateau. If bone cement was used then excess bone cement is then removed after impaction.

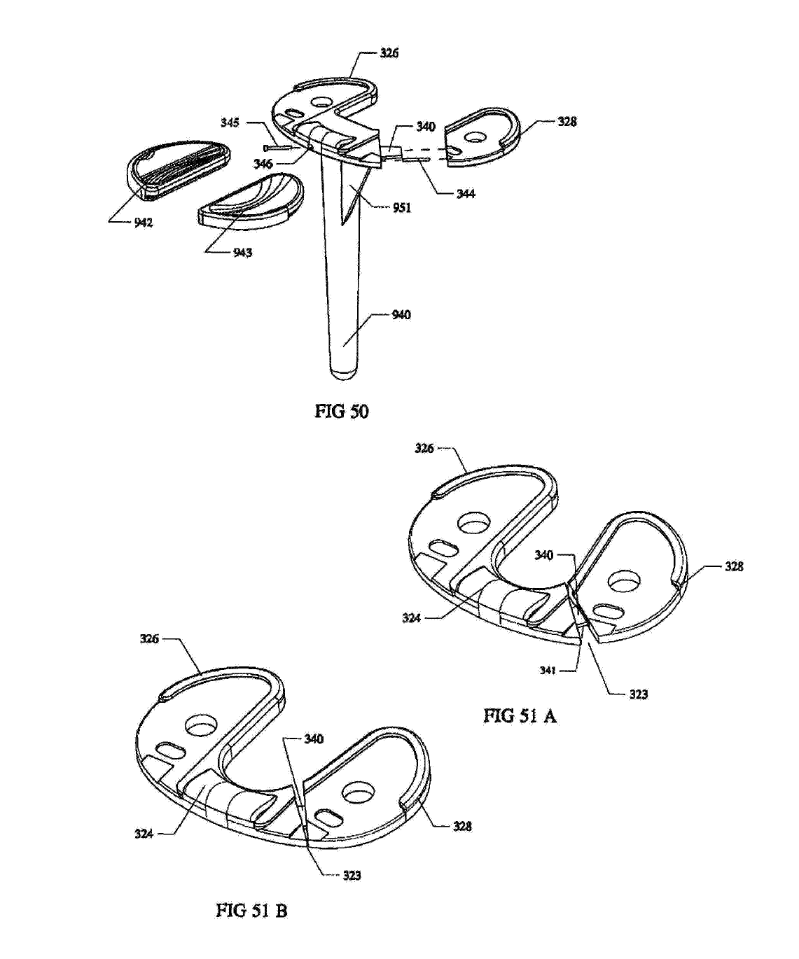

Referring to FIGS. 45 and 46, the stem sub-component is structured with fins 951 that provide rotational stability when engaged with supporting bone and provide support of the baseplate sub-component 941. The under surface of the baseplate sub-component 941 is supported by the proximal surfaces 952 of the fins 951. Alternatively, as shown in FIG. 50, the lateral baseplate sub-component 326 and the stem sub-component 940 may be structured as a unitary sub-component with the medial baseplate sub-component 328 structured to be engaged or joined thereon.

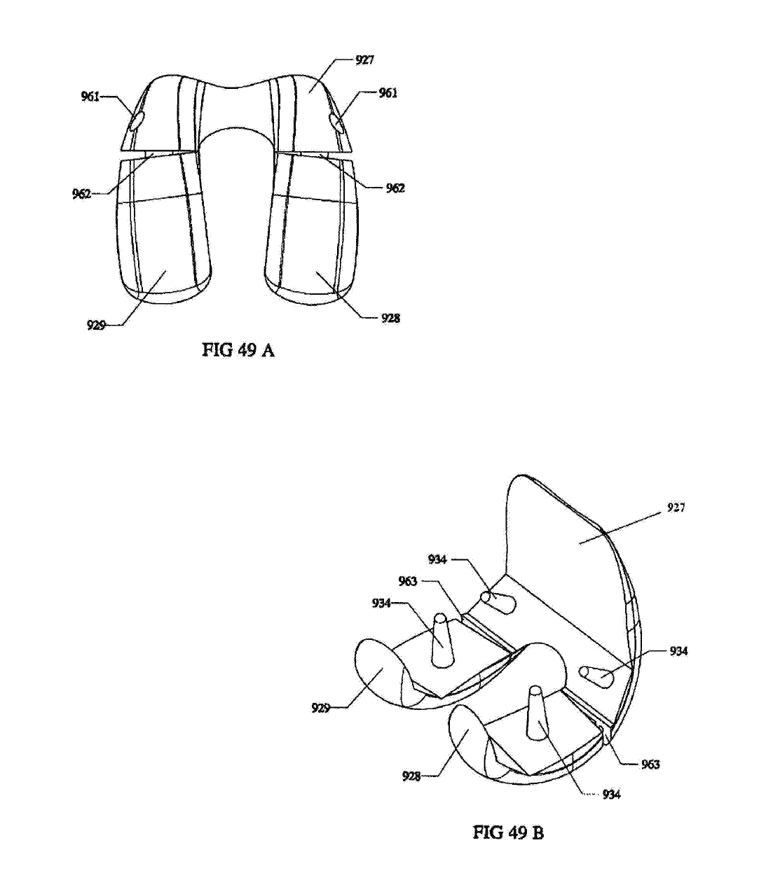

Referring to FIGS. 47 and 48, in another embodiment of the invention a bracket 960 may be used to secure the baseplate sub-component 941 to the stem sub-component 940. The baseplate sub-component 941 is placed onto the stem sub-component 940 as described above with the receiving channel 958 of the baseplate sub-component 941 slidably received by receiving groove 957 in the stem sub-component 940. After the baseplate sub-component 941 has been positioned on the stem sub-component 940 the bracket 960 is placed onto the anterior surface of the stem sub-component in a recessed area 956 and the baseplate sub-component 941 and secured with threaded fasteners 959 placed through receiving clearance holes 954 in the bracket 960 and into threaded receiving holes 955 in the stem sub-component. After securing the baseplate sub-component to the stem sub-component the knee is flexed to greater than 90.degree. to provide access for an impaction tool and the tibial component is impacted onto the tibial plateau. If bone cement was used then excess bone cement is then removed after impaction. The other features and functions of the embodiment shown in FIGS. 47 and 48 are as described above and shown in FIGS. 45 and 46.

As described above, sub-components comprising the femoral and the tibial component are oriented one to the other in forming the femoral and the tibial component, respectively. The process of placing the sub-components into the joint cavity, aligning and orienting them, engaging or joining them one to the other and securing them to supporting bone can be simplified and enhanced through the use of instruments to hold one or more sub-components while placing them into the joint cavity and to hold two or more sub-components properly oriented during assembly or while securing them to supporting bone.

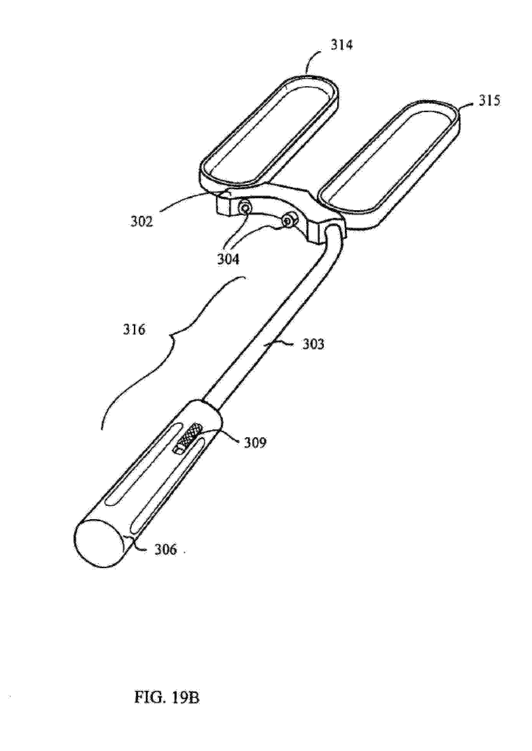

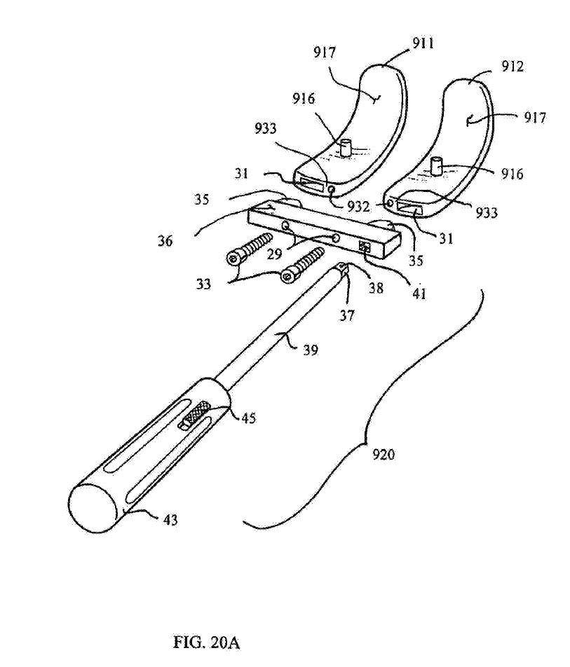

Referring to FIGS. 19 A & B, independent tibial baseplate sub-components 314 and 315 are held in proper orientation one to the other by a baseplate inserter 316. In one embodiment the tibial inserter 316 is comprised of a bracket 302 that spans the baseplate sub-components 314 and 315 along their respective anterior surfaces 317. The respective mating surfaces 308 on the cross bar 302 conform to such anterior baseplate sub-component surfaces 317 to prevent axial rotation of the independent baseplate subcomponents 314 and 315 during placement into the joint cavity. The baseplate sub-components are fastened to the bracket 302 by threaded fasteners 304 placed through clearance holes 305 in the bracket 302 and threaded into threaded receiving holes 301 in the medial 315 and lateral 314 baseplate sub-components. In an alternate embodiment the inserter shaft 303 attaches to the bracket 302 medially anterior to the medial baseplate sub-component 315 allowing for easier placement of the baseplate sub-components 314 and 315 and tibial inserter 316 through a vertical incision running along the medial aspect of the patella. Alternatively, the inserter shaft 303 may be attached midway along the bracket 302 or on the lateral aspect of the bracket 302. In an alternative embodiment of the invention the bracket 302 may be attached to the individual baseplate sub-components with snap-fit connectors, trinkle locks, dove tale connections, or other means to attach two parts together. The inserter shaft 303 may have a quick attach mechanism, such as a trinkle lock 312, structured in a square drive 310, the trinkle lock 312 holding the inserter shaft 303 in the square receiving hole 311, which has a receiving dimple (not shown) to receive the trinkle lock 312, in the bracket 302 while the square drive 310 prevents axial rotation between the inserter shaft 303 and bracket 302. The trinkle lock 312 is normally locked and can be released by pulling back on the release button 309. A detachable inserter shaft 303 is desirable to enable removal of the inserter shaft 303 while leaving the bracket 302 in place to stabilize the individual baseplate sub-components during range of motion assessment or during cementing when it is helpful to allow the incision to close and the patella to track in the trochlea. Alternatively, the inserter shaft 303 may be integral with the bracket 302. In general, the bracket 302 would be available in multiple sizes to accommodate a range of baseplate sub-component sizes and mediolateral spacing. Alternatively, the bracket 302 may be structured to vary in length by including a sliding or telescoping mechanism axially. The baseplate inserter may be made from a suitable metal, such as stainless steal. Optionally, the handle 306 may be made of a suitable plastic, such as acetyl, Ultem, or celcon, or a phenolic material.

In another embodiment of the current invention the bracket 302 may be structured to be implantable in the event additional stability between the medial 315 and lateral 314 baseplate sub-components is beneficial. In which case the bracket 302 and fixation devices, such as screws 304, are made from a suitable implantable material such as titanium, titanium alloy, stainless steel, cobalt chromium alloy; or from a suitable polymer such as PEEK or polyethylene.

In one method of use in which the baseplate sub-components 314 and 315 are to be secured to supporting bone with bone cement, the medial baseplate sub-component 315 is first attached to the bracket 302. Trial femoral sub-components (not shown) are placed on the lateral and medial femoral condyles. Bone cement is applied to the underside of the baseplate sub-components 314 and 315, and the independent lateral baseplate 314 is placed into the lateral compartment of the knee. The medial baseplate 315 is placed into the medial compartment with the aid of the tibial inserter 316 until a threaded fastener 304 can be passed through receiving hole 305 in the bracket 302 and into the threaded receiving hole 301 in the lateral baseplate sub-component 314. Trial insert bearings (not shown) are placed on the baseplate sub-components 314 and 315, and the knee is extended to provide a compressive force to the tibial components. Optionally, the tibial inserter 316 may be structured with an alignment guide to reference the mechanical axis of the knee to aid in aligning the tibial components. Alternatively, the tibial inserter 316 may be structured with a navigational tracker to enable surgical navigation of the tibial inserter 316 and the attached baseplate sub-components 314 and 315 for proper alignment within the joint cavity. The inserter shaft 303 may be removed and the bracket 302 left in place to improve access to the joint cavity for cement cleanup. The inserter shaft 303 may be removed by pulling back on the trinkle lock release button 309. Once the cement has set the bracket 302 is removed.

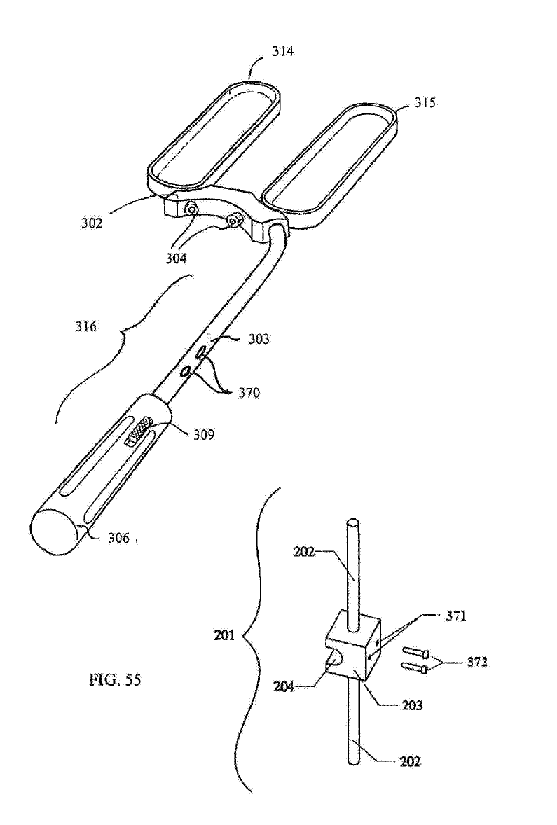

Optionally, the tibial inserter 316 may be structured for attachment of an alignment guide. Referring to FIG. 55, an alignment guide 201 with an alignment rod 202 may be used to check alignment of the tibial baseplate sub-components 314 and 315 relative to the mechanical axis of the leg by attaching the alignment guide 201 to the tibial inserter 316, such attachment structured as a channel 204 in the base 203 of the alignment guide 201 that slidably fits over the shaft 303 to stabilize the alignment guide 201 in proper alignment relative to the tibial inserter 316. The alignment guide is attached to the tibial inserter by threaded fasteners 372 passed through clearance receiving holes 371 in the base 203 and threaded into threaded receiving holes 370 in the inserter shaft 303. Tibial sub-component 314 and 315 alignment is checked with the alignment guide 201 attached to the tibial inserter 316 and the tibial sub-components placed on the prepared tibial resections. Femoral trials and trial insert bearings are placed and the knee is extended to full extension. When properly aligned, the alignment rod 202 passes over the hip joint center, the knee joint center and the ankle center.

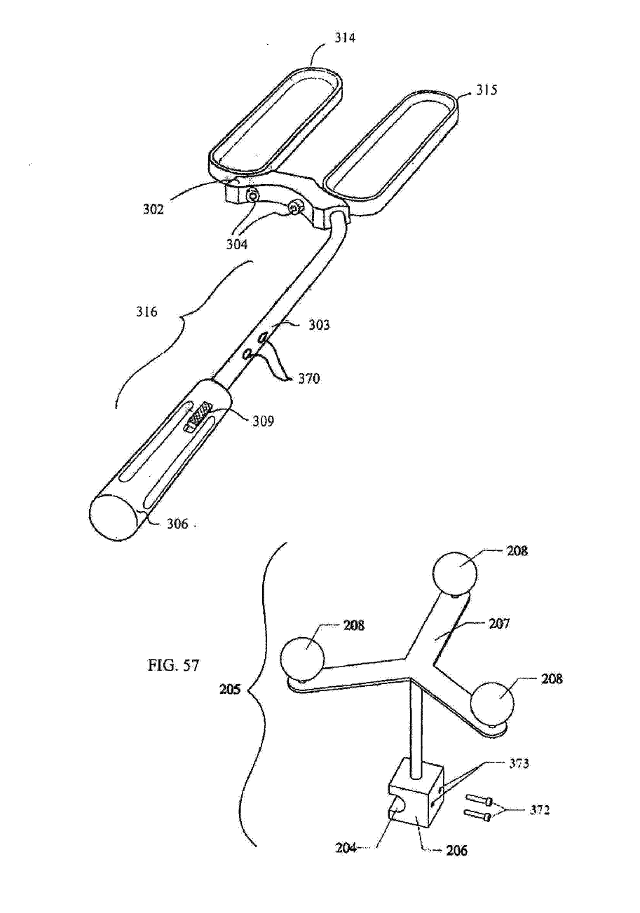

Optionally, the tibial inserter 316 may be structured for attachment of a surgical navigation tracker for use with a surgical navigation system. Referring to FIG. 57, a surgical navigation tracker 205 with three reflective spheres 208 supported on a frame 207 and a base 206 may be used to check alignment of the tibial baseplate sub-components 314 and 315 relative to the mechanical axis of the leg by attaching the a surgical navigation tracker 205 to the tibial inserter 316, such attachment structured as a channel 204 in the base 206 of the a surgical navigation tracker 205 that slidably fits over the shaft 303 to stabilize the a surgical navigation tracker 205 in proper alignment relative to the tibial inserter 316. The a surgical navigation tracker 205 is attached to the tibial inserter by threaded fasteners 372 passed through clearance receiving holes 373 in the base 206 and threaded into threaded receiving holes 370 in the inserter shaft 303. Tibial sub-component 314 and 315 alignment is checked with the a surgical navigation tracker 205 attached to the tibial inserter 316 and the tibial sub-components placed on the prepared tibial resections. Femoral trials and trial insert bearings are placed and the knee is extended to full extension. The surgical navigation system will measure knee alignment and provide a report to the surgeon. Alternatively, the alignment guide 201 and the surgical navigation tracker 205 may be structured for attachment to the tibial inserter 316 with “T” slots; dovetail locks; cylindrical interlocks; button interlocks; spherical interlocks; or a combination of these, or other connecting means used to connect two or more parts.

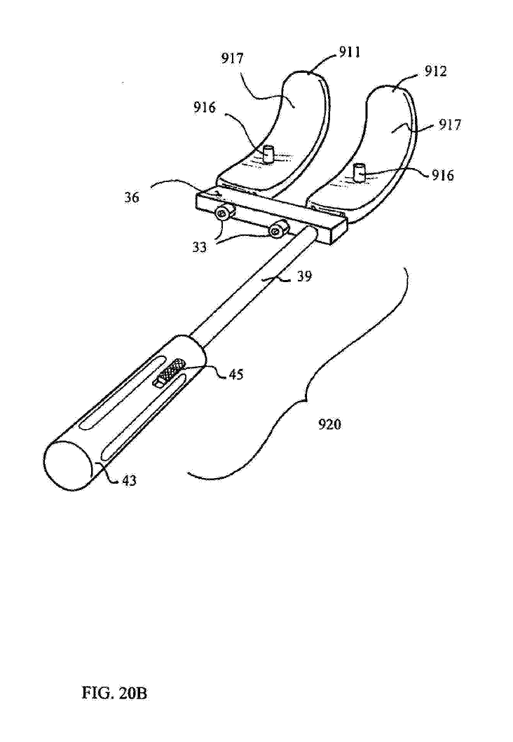

As described above, one embodiment for the femoral articular surfaces is to resurface the medial and lateral tibiofemoral compartments and the patellofemoral compartment; there is benefit in staging implantation of the components if bone cement is used to secure the implants to supporting bone. Referring to FIGS. 20 A and B, the independent medial 912 and lateral 911 condylar sub-components may be cemented in place before the trochlear sub-component. In one embodiment of the present invention these condylar sub-components are oriented one to the other by a femoral inserter 920 for placement into the joint cavity. In one embodiment the femoral inserter 920 is comprised of a bracket 36 that spans the medial 912 and lateral 911 condylar sub-components along their respective anterior surfaces 933. The bracket 36 is structured with protruding tabs 35 that slidably fit into receiving pockets 31 in the medial 912 and lateral 911 condylar sub-components to prevent axial rotation of each condylar sub-component, respectively, during placement into the joint cavity. The condylar sub-components 911 and 912 are fastened to the bracket 36 by threaded fasteners 33 placed through clearance holes 29 in the bracket 36 and threaded into threaded receiving holes 932 in the individual condylar sub-components 911 and 912. In an alternate embodiment the inserter shaft 39 attaches to the bracket 36 medially anterior to the medial condylar sub-component 912 allowing for easier placement of the condylar sub-components 911 and 912 and femoral inserter 920 through a vertical incision running along the medial aspect of the patella. Alternatively, the inserter shaft 39 may be attached midway along the bracket 36 or on the lateral aspect of the bracket 36. In an alternative embodiment of the invention the bracket 36 may be attached to the individual condylar sub-components with snap-fit connectors, trinkle locks, dove tale connections, or other means to attach two parts together. The inserter shaft 39 may have a quick attach mechanism, such as a trinkle lock 38, structured in a square drive 37, the trinkle lock 38 holding the inserter shaft 39 in the square receiving hole 41, which has a receiving dimple (not shown) to receive the trinkle lock 38, in the bracket 36 while the square drive 37 prevents axial rotation between the inserter shaft 39 and bracket 36. The trinkle lock 38 is normally locked and can be released by pulling back on the release button 45. A detachable inserter shaft 39 is desirable to enable removal of the inserter shaft 39 while leaving the bracket 36 in place to stabilize the individual condylar sub-components 911 and 912 during range of motion assessment or during cementing when it is helpful to allow the incision to close and the patella to track in the trochlea. Alternatively, the inserter shaft 39 may be integral with the bracket 36. In general, the bracket 36 would be available in multiple sizes to accommodate a range of condylar sub-component sizes and mediolateral spacing. Alternatively, the bracket 36 may be structured to vary in length by including a sliding or telescoping mechanism axially. The femoral inserter may be made from a suitable metal, such as stainless steal. Optionally, the handle 43 may be made of a suitable plastic, such as acetyl, Ultem, or celcon, or a phenolic material.

In one method of use in which bone cement is used to secure the femoral component to supporting bone, the first step is to prepare receiving holes in the distal femur for the posts 916 on the independent condylar sub-components 911 and 912. A drill and drill guide (not shown) are used to prepare receiving holes in the femoral condyles for the posts 916 on the medial and lateral condylar sub-components. Optionally, the lateral condylar sub-component is attached to the insertion tool 920 outside the joint cavity. Cement is applied to the prepared medial and lateral condyles and to the inner surfaces 917 of the medial 912 and lateral 911 condylar sub-components. The medial condylar sub-component 912 is placed onto the medial condyle and the insertion tool 920 is used to place the lateral condylar sub-component 911 under the patellar ligament and into the lateral tibiofemoral compartment. When the lateral condylar sub-component is in place, the insertion tool 920 is assembled to the medial condylar sub-component by advancing a threaded fastener 33 into the receiving hole 932 in the sub-component. The medial and lateral tabs 35 protruding from the bracket 36 engage the medial and lateral condylar sub-components, respectively, by fitting into conforming pockets 31 therein. The shape and cross section of such tabs 35 being structured to accommodate various receiving pockets in the condylar sub-components as described below. Trial tibial baseplate sub-components and trial tibial inserts (not shown) are placed onto the prepared lateral and medial tibial plateaus. Optionally, the inserter shaft 39 is structured to receive an alignment guide to reference the mechanical axis of the femur and tibia to aid in aligning the condylar sub-components 911 and 912. The knee is extended to load the implants. Excess bone cement is removed. The inserter handle 43 and inserter shaft 39 may be removed and the bracket 36 left in place to improve access to the joint cavity for cement cleanup and to check range of motion and tissue balance.

The inserter handle 43 and inserter shaft 39 are removed by pulling back on the trinkle release button 45 which releases the trinkle lock 38 connecting the inserter shaft 39 to the bracket 36 in the square receiving hole 41 in the bracket 36. After the bone cement has set the bracket 36 is removed. The trochlear sub-component 910, FIG. 21, is now implanted in similar fashion by first preparing a receiving hole for the post 916 on the inner surface of the trochlear sub-component 910 using a drill and drill guide (not shown), placing bone cement onto the prepared femoral trochlea and onto the inner surface 917 of the trochlear sub-component, shown in FIG. 21. Referring to FIGS. 34 A and B, the two bosses 450 protruding from the posterior interface surfaces 461 of the trochlear sub-component 910 are structured for each boss 450 to engage a condylar sub-component 911 or 912 in a respective receiving pocket 31 in the anterior interface surface 462 of each condylar sub-component 911 or 912 to properly orient the trochlear sub-component 910 to the condylar sub-components 911 and 912. The trochlear sub-component is then impacted onto the femoral trochlea establishing kinematic positioning of the trochlear sub-component. A contoured impactor (not shown) is used to seat the trochlear sub-component. After impaction the excess bone cement is removed. The patellar component or patellar trial is placed onto the patella and the knee is flexed and extended to assess range of motion and soft tissue balance checked.

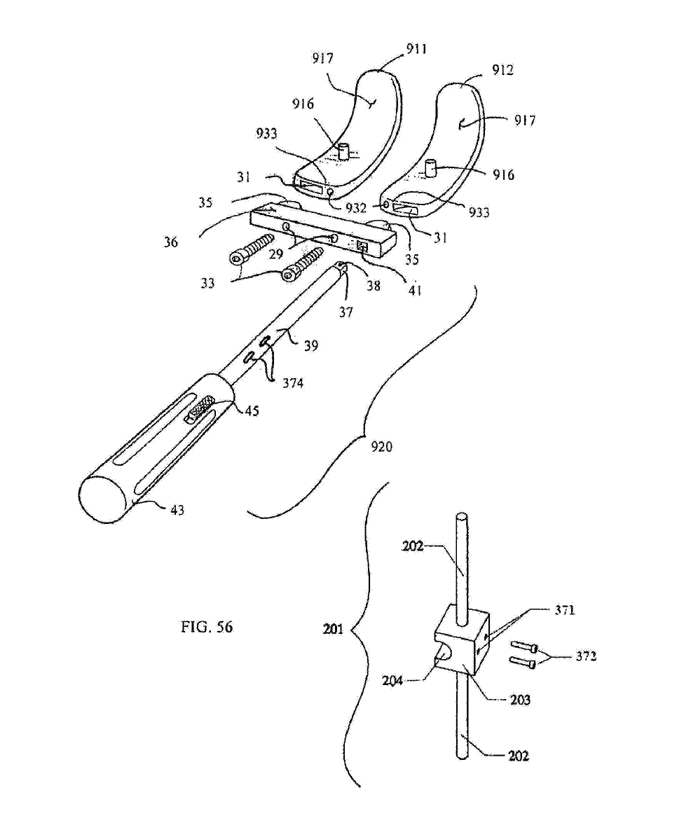

Optionally, the femoral inserter 920 may be structured for attachment of an alignment guide. Referring to FIG. 56, an alignment guide 201 with an alignment rod 202 may be used to check alignment of the femoral condylar sub-components 911 and 912 relative to the mechanical axis of the leg by attaching the alignment guide 201 to the femoral inserter 920, such attachment structured as a channel 204 in the base 203 of the alignment guide 201 that slidably fits over the shaft 39 to stabilize the alignment guide 201 in proper alignment relative to the femoral inserter 920. The alignment guide is attached to the femoral inserter by threaded fasteners 372 passed through clearance receiving holes 371 in the base 203 and threaded into threaded receiving holes 374 in the inserter shaft 39. Femoral condylar sub-component 314 and 315 alignment is checked with the alignment guide 201 attached to the femoral inserter 920 and the femoral condylar sub-components placed on the prepared femoral resections. Tibial baseplate trials and trial insert bearings are placed and the knee is extended to full extension. When properly aligned, the alignment rod 202 passes over the hip joint center, the knee joint center and the ankle center.

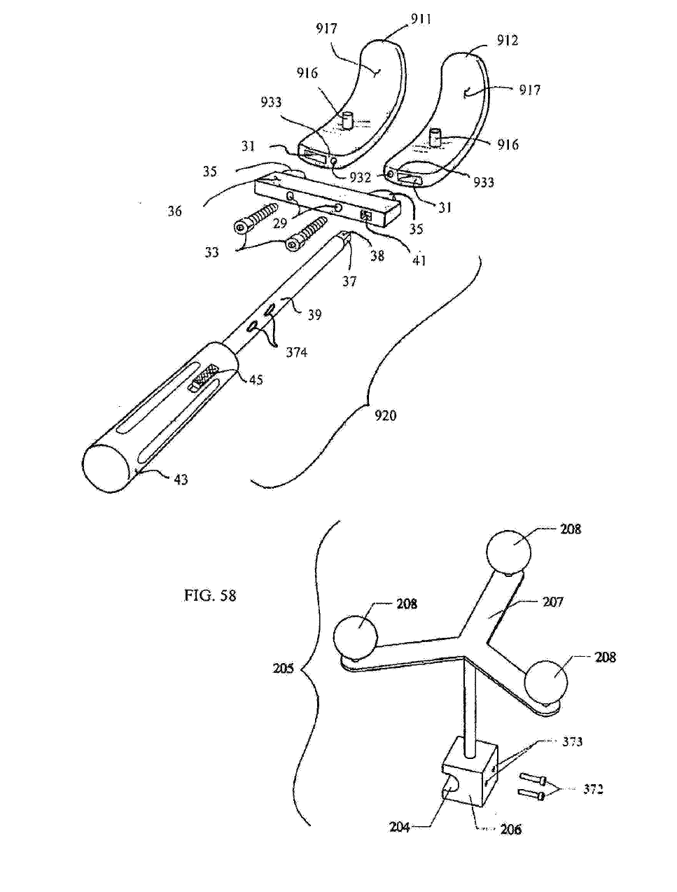

Optionally, the femoral inserter 920 may be structured for attachment of a surgical navigation tracker for use with a surgical navigation system. Referring to FIG. 58, a surgical navigation tracker 205 with three reflective spheres 208 supported on a frame 207 and a base 206 may be used to check alignment of the femoral condylar sub-components 911 and 912 relative to the mechanical axis of the leg by attaching the a surgical navigation tracker 205 to the femoral inserter 920, such attachment structured as a channel 204 in the base 206 of the a surgical navigation tracker 205 that slidably fits over the shaft 39 to stabilize the a surgical navigation tracker 205 in proper alignment relative to the femoral inserter 920. The a surgical navigation tracker 205 is attached to the femoral inserter by threaded fasteners 372 passed through clearance receiving holes 373 in the base 206 and threaded into threaded receiving holes 374 in the inserter shaft 39. Femoral condylar sub-component 911 and 912 alignment is checked with the a surgical navigation tracker 205 attached to the femoral inserter 920 and the femoral condylar sub-components 911 and 912 placed on the prepared tibial resections. Tibial baseplate trials and trial insert bearings are placed and the knee is extended to full extension. The surgical navigation system will measure knee alignment and provide a report to the surgeon. Alternatively, the alignment guide 201 and the surgical navigation tracker 205 may be structured for attachment to the femoral inserter 920 with “T” slots; dovetail locks; cylindrical interlocks; button interlocks; spherical interlocks; or a combination of these, or other connecting means used to connect two or more parts.

Additional components or steps as known to those skilled in the art may be performed within the scope of the invention. Further, one or more of the listed steps or components need not be performed in a procedure within the scope of the present invention. While a select embodiments of the present invention have been described, it should be understood that various changes, adaptations and modifications may be made therein without departing from the spirit of the invention and the scope of the appended claims.

What is claimed:

1. An apparatus for replacing the surfaces of a joint between a first bone and a second bone, the first bone moving in a predetermined manner with a second bone, the apparatus comprising
a first bone arthroplasty;
a second bone arthroplasty, at least one of said first bone arthroplasty or second bone arthroplasty including a plurality of first bone sub-components or a plurality of second bone baseplate sub-components structured for mimicking and replacing a bearing surface of the first bone or second bone; and
a flexible interconnect mechanism for slidably interconnectedly receiving two adjacent first bone sub-components or two adjacent second bone baseplate sub-components,
wherein each of said adjacent interconnected first bone sub-components or said adjacent interconnected second bone baseplate sub-components have relative angular motion one to the other, and further wherein upon assembly and when fully assembled the relative angular motion between adjacent interconnected first bone sub-components or adjacent interconnected second bone baseplate sub-components is partially constrained.

2. The apparatus of claim 1 wherein said apparatus is a knee replacement, wherein the first bone arthroplasty is structured for attachment to a distal surface of a femur and said second bone arthroplasty is structured for attachment to a proximal surface of a tibia.

3. An apparatus for replacing the surfaces of a joint between a first bone and a second bone, the first bone moving in a predetermined manner with a second bone, the apparatus comprising:
a first bone arthroplasty including a plurality of first bone sub-components structured for mimicking and replacing a bearing surface of the first bone, wherein each of said plurality of first bone sub-components has an inner surface structured to be secured to the first bone and an outer surface; and
a second bone arthroplasty including a unitary baseplate or a plurality of baseplate sub-components structured for mimicking and replacing a bearing surface of the second bone; and
at least one flexible interconnect mechanism structured to slidably interconnectedly receive two adjacent first bone sub-components or two adjacent second bone baseplate sub-components,
wherein the outer surface of said plurality of first bone sub-components contact said second bone arthroplasty and further wherein each of said adjacent interconnected first bone sub-components or said adjacent interconnected second bone baseplate sub-components have relative angular motion one to the other and are structured to be partially constrained upon assembly and partially constrained when fully assembled, the resulting configuration of the first bone arthroplasty and second bone arthroplasty articulating in a predetermined manner to restore proper kinematics.

4. The apparatus of claim 3 wherein the second bone baseplate sub-components comprise a plurality of tibial sub-components.

5. The apparatus of claim 3 wherein the first bone arthroplasty includes a plurality of femoral sub-components.

6. The apparatus of claim 3 wherein at least one of the plurality of second bone baseplate sub-components includes a threaded receiving hole on an anterior surface thereof for receiving an alignment instrument in mating relationship.

7. The apparatus of claim 3 wherein at least one of the plurality of first bone sub-components includes a conforming pocket and a threaded receiving hole on a surface thereof for receiving an insertion instrument in mating relationship.

8. The apparatus of claim 6 wherein the alignment instrument comprises a bracket, said bracket including at least one contact surface structured for contacting at least one of the plurality of second bone baseplate sub-components and at least one threaded fastener for fastening said bracket in said at least one threaded receiving hole, said bracket contoured for a fully constrained lock between the bracket and said at least one of the plurality of second bone baseplate sub-components.

9. The apparatus of claim 7 wherein the insertion instrument comprises a bracket, said bracket including (a) at least one contact surface structured for contacting said at least one of the plurality of first bone sub-components; (b) at least one medial and lateral boss extending outwardly from said bracket for insertion into said at least one conforming pocket; and (c) at least one threaded fastener for fastening said bracket in said threaded receiving hole, said bracket contoured for a fully constrained lock between the bracket and the plurality of first bone sub-components.

10. The apparatus of claim 3 wherein said flexible interconnect mechanism is structured to be slidably received in a receiving groove of adjacent first bone sub-components.

11. The apparatus of claim 3 wherein said flexible interconnect mechanism is structured to be slidably received in a receiving groove of adjacent second bone baseplate sub-components.

12. The apparatus of claim 1 wherein each of the said plurality of first bone sub-components has an inner surface structured to be secured to the first bone and an outer surface structured to contact said second bone arthroplasty.

13. The apparatus of claim 1 wherein said second bone baseplate sub-components comprise a plurality of tibial sub-components.

14. The apparatus of claim 1 wherein said first bone arthroplasty includes a plurality of femoral sub-components.

15. The apparatus of claim 1 wherein at least one of said plurality of second bone baseplate sub-components includes at least one threaded receiving hole on an anterior surface thereof for receiving an alignment instrument in mating relationship.

16. The apparatus of claim 1 wherein at least one of said plurality of first bone sub-components includes at least one conforming pocket and at least one threaded receiving hole on a surface thereof for receiving an insertion instrument in mating relationship.

17. The apparatus of claim 15 wherein the alignment instrument comprises a bracket, said bracket including at least one contact surface structured for contacting said at least one of the plurality of second bone baseplate sub-components and at least one threaded fastener for fastening said bracket in said at least one threaded receiving hole, said bracket contoured for a fully constrained lock between the bracket and said at least one of the plurality of second bone baseplate sub-components.

18. The apparatus of claim 16 wherein the insertion instrument comprises a bracket, said bracket including (a) at least one contact surface structured for contacting at least one of the plurality of first bone sub-components; (b) at least one medial and lateral boss extending outwardly from said bracket for insertion into said at least one conforming pocket; and (c) at least one threaded fastener for fastening said bracket in said at least one threaded receiving hole, said bracket contoured for a fully constrained lock between the bracket and said at least one of the plurality of first bone sub-components.

19. The apparatus of claim 1 wherein said flexible interconnect mechanism is structured to be slidably interconnectedly received in a receiving groove of an adjacent first bone sub-component.

20. The apparatus of claim 1 wherein said interconnect mechanism is structured to be slidably interconnectedly received in a receiving groove of an adjacent second bone baseplate sub-component.

21. An apparatus for replacing the surfaces of a joint between a first bone and a second bone, the first bone moving in a predetermined manner with a second bone, the apparatus comprising
a first bone arthroplasty;
a second bone arthroplasty, at least one of said first bone arthroplasty or second bone arthroplasty including a plurality of first bone sub-components or a plurality of second bone baseplate sub-components structured for mimicking and replacing a bearing surface of the first bone or second bone; and
a flexible interconnect mechanism for slidably interconnectedly receiving two adjacent first bone sub-components or two adjacent second bone baseplate sub-components,
wherein each of said adjacent interconnected first bone sub-components or said adjacent interconnected second bone baseplate sub-components have relative angular motion one to the other when fully assembled and wherein the relative angular motion between adjacent interconnected first bone sub-components or adjacent interconnected second bone baseplate sub-components is a flex-interface.
